(19) United States
(12) Patent Application Publication
(10) Pub. No.: US 2018/0242844 A1
(43) Pub. Date: Aug. 30, 2018

(54) SYSTEMS AND METHODS FOR FUNCTIONAL OPTICAL COHERENCE TOMOGRAPHY

(71) Applicant: Northwestern University, Evanston, IL (US)

(72) Inventors: Wenzhong LIU, Evanston, IL (US); Hao F. ZHANG, Deerfield, IL (US); Kieren J. PATEL, Santa Monica, CA (US)

(21) Appl. No.: 15/751,107

(22) PCT Filed: Aug. 5, 2016
(86) PCT No.: PCT/US16/45791

§ 371 Date: Feb. 7, 2018

Related U.S. Application Data
(60) Provisional application No. 62/202,617, filed on Aug. 7, 2015.

(51) Int. Cl. A61B 3/12; A61B 3/10; A61B 5/1455; A61B 3/00; G06T 7/00; G16H 40/63

(52) U.S. Cl. CPC A61B 3/1233; A61B 3/102; A61B 5/14555; A61B 3/0025; G06T 2207/30041; G16H 40/63; A61B 2576/02; G06T 2207/10101; G06T 2207/30104; G06T 7/0012

(57) ABSTRACT

The present disclosure provides systems and methods for objective focal length free measurements of fluid flow using OCT. In certain disclosed examples, fOCT data is acquired and optical information is extracted from fOCT scans to quantitatively determine a flow rate of fluid in the target. Determinations of flow rate can enable determination of a change in rate of an analyte over time. The current methods and systems of the disclosure can be used in assessing metabolism of a tissue, where oxygen is the analyte detected, or other functional states, and, more generally, be used for the diagnosis, monitoring and treatment of disease.

### CROSS-REFERENCE

This application claims the benefit of priority to PCT Patent Application No. PCT/US16/45791, titled “Systems And Methods For Functional Optical Coherence Tomography,” filed on Aug. 5, 2016, which claims the benefit of priority to U.S. Patent Application Ser. No. 62/202,617, titled “Systems and Methods for Functional Optical Coherence Tomography”, filed on Aug. 7, 2015, each of which is incorporated herein by reference in its entirety for all purposes.

### STATEMENT REGARDING FEDERALLY SPONSORED RESEARCH FOR DEVELOPMENT

This invention was made with government support under 1 R01 EY019951 and 1 R24 EY022883 awarded by the National Institutes of Health and CBET-1055379 and DBI-1353952 awarded by the National Science Foundation. The government has certain rights in the invention.

### BACKGROUND OF THE DISCLOSURE

Optical coherence tomography (OCT) is a non-invasive optical imaging technique which produces depth-resolved reflectance imaging of samples through the use of a low coherence interferometer system. OCT imaging allows for three-dimensional (3D) visualization of structures in a variety of biological systems and non-biological systems not easily accessible through other imaging techniques. In some instances, OCT may provide a non-invasive, non-contact approach to assess information without disturbing or injuring a target or sample. In some examples, function optical coherence tomography (fOCT) can provide additional information regarding physical and chemical attributes inside vessels and structures, such as measurements of fluid flow. In medical applications, fOCT measurements can be used for diagnostic or monitoring purposes of a variety of fluids in the treatment of various diseases.

In ophthalmic applications, previous experimental investigations have focused on the measurement of retinal blood flow rate with various types of OCT, such as spectral domain optical coherence tomography (SD-OCT), for the diagnosis and monitoring of various ocular diseases. In some examples, to measure blood flow, both the vessel size and the blood velocity must be quantified. For example, using SD-OCT, retinal vessel size is normally extracted from a cross-sectional OCT B-scan image, due to circumstances in which axial resolution may be higher than lateral resolution with this type of OCT. Several techniques have been proposed to extract retinal blood flow velocity: (1) the indirect method which employs first calculating the phase shift variance or light intensity within vessels, and then calibrating the measured results using well-controlled phantoms; and (2) the mean phase shift method, in which the phase shift between two adjacent A-lines can be used to directly quantify axial flow velocity. However, this latter method requires the Doppler angle (i.e., the angle between the probing beam and retinal vessels) to measure absolute velocity.

For either method, the Doppler angle must be either measured or circumvented in calculating fluid flow velocity. While methods exist to solve the issue of the Doppler angle in these calculations, such as the multiple-beams scanning scheme, or the en face Doppler approach, such methods require the objective focal length between the objective and the target. In ophthalmic applications, where an eyeball is scanned using OCT, this objective focal length may include the axial length of the eyeball, wherein the retina is the target or subject. The multiple-beams scanning approach requires the eyeball axial length in order to access the geometrical information of the retinal vessels, which thus enables the Doppler angles calculation. The en face Doppler method requires the eyeball axial length to calibrate a transverse scanning dimension to measure flow rate.

For ophthalmic applications, instead of measuring the eyeball axial length for every subject, a more commonly applied approach may be use of a universal empirical eyeball axial length for flow quantification. Commonly this distance is fixed as an assumption in calculations of fluid flow velocity. In reality however, eyeball axial length may vary with age (e.g., the aged eye may have a significantly shorter axial length), vary with other eye conditions or vary due to anatomical variance between individuals. In patients with myopia, for instance, the eyeball axial length of subjects with −4.36 D nearsightedness can be 5.2 mm shorter than subjects with −6.00 D. The use of a universally assumed path length, as in the case of assuming a universal eyeball axial length for all patients or subjects, can affect the accuracy the flow velocity determination. There is need in the art to improve the accuracy of quantifying fluid flow using fOCT, where the objective focal length is unknown or no empirical measurement is possible, such as in measuring retinal blood flow in the eye without knowing or previously measuring the axial length of the eyeball.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of methods and systems of this disclosure are set forth with particularity in the appended claims. A better understanding of the features and advantages of this disclosure will be obtained by reference to the following detailed description that sets forth illustrative examples, in which the principles of the methods and systems of this disclosure are utilized, and the accompanying drawings of which:

FIG. 1 is a schematic of an eyeball and various lengths that may include the axial length of the eye.

FIG. 2a is a schematic of a free space vis-OCT device or system.

FIG. 2b shows an illumination spectrum of a free space vis-OCT device or system.

FIG. 2c shows theoretical and axial resolutions of a free space vis-OCT device or system.

FIG. 2d is a system block diagram of scanning acquisition and analysis of OCT scan functions.

FIG. 3 is a schematic showing data acquisition and data process elements for objective focal length free fluid flow measurement.

FIG. 4 shows process elements for segmenting vessels in a target for objective focal length free fluid flow measurement.

FIG. 5 is a schematic of dual beam scanning. The target is illuminated with concentric circles of first and second beams of radiation concurrently or sequentially.

FIG. 6 is a schematic of the experimental setup for in vivo rodent and phantom imaging

FIG. 7a a light source spectrum for OCT.

FIG. 7b a schematic showing dependence of measured velocity and measured cross-sectional vessel size on the Doppler angle.

FIG. 8 is a schematic of the geometry to quantify the retinal blood flow rate from the dual-ring scanning

FIG. 9a is a data graph showing validation of the flow measurement in phantom experiments.

FIG. 9b is a data graph showing validation of the flow measurement in phantom experiments.

FIG. 10a shows in vivo imaging of retinal blood flow in rodents.

FIG. 10b shows a phase image from the inner-circle scan.

FIG. 11a shows phase stability across a different number of cumulative frames for two vessels, indicated by the red and blue arrows in FIG. 10b.

FIG. 11b shows the estimated Doppler angles for all the vessels from both small and large scanning locations.

FIG. 12a shows recorded rat electrocardiogram during imaging.

FIG. 12b shows the measured pulsatile flow from one retinal artery, highlighted by the red arrow in FIG. 10b.

FIG. 13a shows the measured pulsatile flow from one retinal vein, highlighted by the blue arrow in FIG. 10b.

FIG. 13b shows data consistency in flow imaging of the same rodent subject over seven days.

FIG. 14a is an image of raw OCT data.

FIG. 14b is OCT data after median filtering.

FIG. 14c is mean value subtraction of OCT data.

FIG. 14d is a graph of coefficient of horizontal standard deviation, dash line indicates which region is selected for segmentation.

FIG. 14e is selected regions containing retinal vessels within a B-scan.

FIG. 15 is a schematic of an OCT device or system or probe illuminating a target or subject with a vessel in which there is a fluid flow.

FIG. 16 is a block diagram illustrating a first example architecture of a computer system that can be used in connection with a fOCT device or system.

FIG. 17 is a diagram showing a network with a plurality of computer systems, and a plurality of cell phones and personal data assistants configured with a fOCT device or system.

FIG. 18 is a diagram illustrating a first example a computer system that can be used in connection with one or more fOCT device or systems, including handheld or mobile devices.

FIG. 19 is an example fOCT data processing system.

FIG. 20a is an example of a contour C₁.

FIG. 20b is an example of contour C₂.

The following detailed description of certain examples of the present invention will be better understood when read in conjunction with the appended drawings. For the purpose of illustrating the invention, certain examples are shown in the drawings. It should be understood, however, that the present invention is not limited to the arrangements and instrumentality shown in the attached drawings.

### DETAILED DESCRIPTION OF THE DISCLOSURE

### I. General Overview

The methods and systems of the present disclosure provide for the determination of fluid flow using fOCT. Generally, determination of fluid flow and fluid flow rates using fOCT may include a non-invasive, non-contact method for determining a functional state of target, such as the health of bodily tissue. In some examples, fOCT objective focal length free measurements may be used for determining the change in metabolism of a tissue, therefore indicating something about disease state or health.

Generally, fOCT employs any method of OCT, as known in the art. fOCT objective focal length free measurements provides a method of determining flow rates of fluid without knowing or measuring the objective focal length. As previously described, such as in U.S. Pat. No. 9,046,339 and U.S. Pat. No. 8,244,334, the methods of which are incorporated by reference herein, determining fluid flow using OCT often requires measurement of the objective focal length before OCT scanning, or an assumed universal length is used for fluid flow calculations. Measurements of the objective focal length, in some examples the axial length of an eyeball for ophthalmic applications, can be cumbersome or impossible. A universal assumption of the objective focal length, such as a universal assumption of the axial length of the eye can also lead to inaccurate results. The methods and systems of the present disclosure determine fluid flow using OCT without need for objective focal length measurement, predetermination or assumption of length.

In some examples, the accurate quantification of a fluid flow can be used to determine a change in state of target function. For example, a change in metabolism or oxygen consumption of the human retina may indicate retinal disease. In the case of skin monitoring and glucose in sweat, blood glucose levels for diabetic monitoring may be performed using the methods, devices and systems of the disclosure.

Given the label-free, non-invasive, non-contact methods of the disclosure, a variety of medical applications may be employed including the disease monitoring and diagnosis of cancer and variety of other ocular diseases.

### II. Brief Description

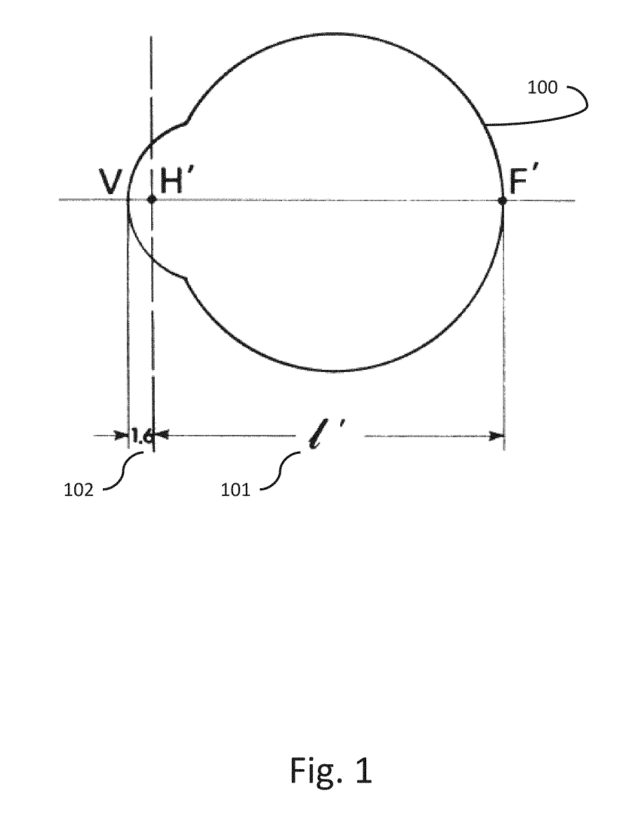

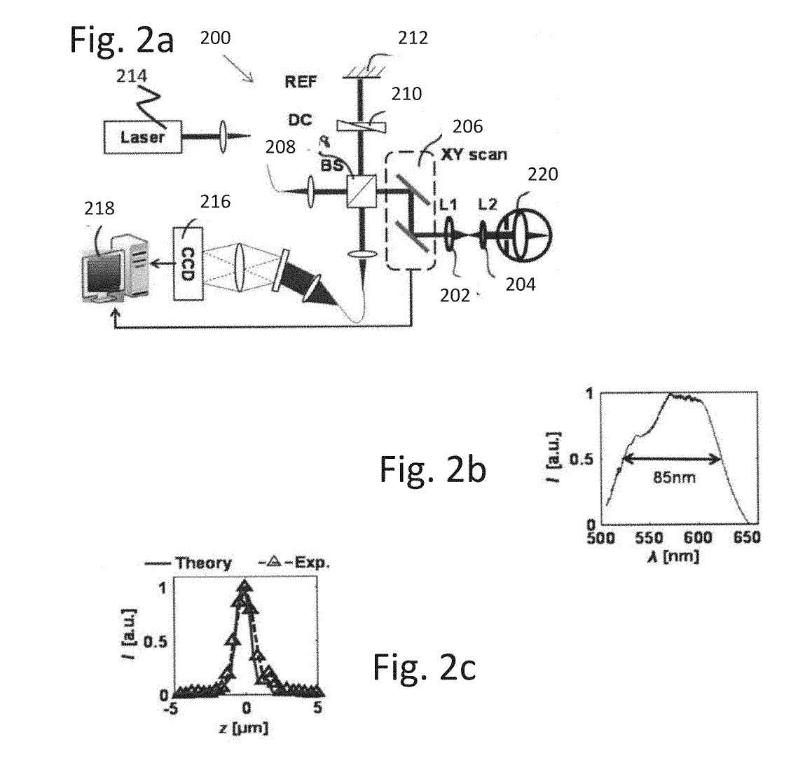

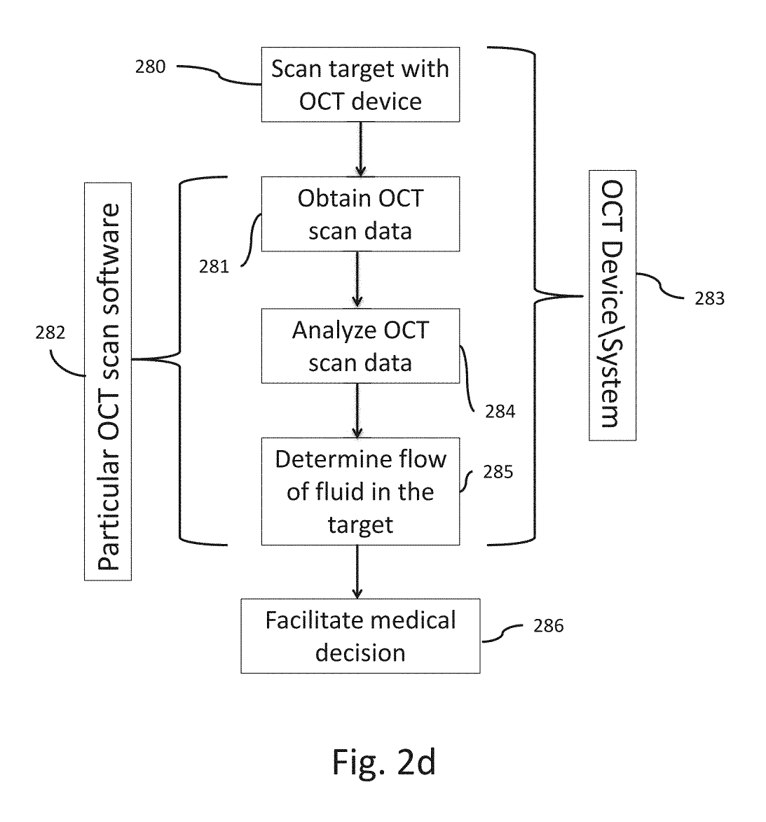

An example eyeball is illustrated in FIG. 1, which shows various lengths including an axial length. In some examples, the methods, device and systems of the present disclosure provide for OCT data acquisition and OCT data analysis of a target, such as the eye 100, as shown in FIG. 2d. First, the target may be scanned with an OCT device to generate OCT scans, 280. One or more OCT scans may be generated and obtained by one or more components of the OCT device or system, such as a spectrometer, 281. OCT scan data may be analyzed by a logic program, software, algorithm or computer, 284. Based on the analysis performed by a logic program, software, algorithm or computer, fluid flow in the target may be determined, 285. In some cases, where the fluid flow is bodily fluid flow, and the target is a bodily tissue, the determining of bodily fluid flow may be used to facilitate a medical decision, 286. In some examples, the OCT device or system, 283, may be configured with both hardware components for generating OCT scans of a target and software, 282, to perform data analysis.

In a first aspect, the present disclosure provides for a method for imaging and quantifying fluid flow in a subject. In some aspects, the method is achieved by acquiring a first OCT data set for a first series of transverse locations in the subject. The first data set may include a first plurality of measurements, and wherein at least two of the first plurality of measurements are made within a region substantially near a transverse location in the first series of transverse locations. The first data set may be acquired with a first beam of radiation having a first angle with respect to the subject. This is followed by acquiring a second data set for a second series of transverse locations in the sample. The second data set may include a second plurality of measurements, wherein at least two measurements of the second plurality of measurements are made within a predetermined distance from the same transverse location as the at least two measurements of the first plurality of measurements. The second data may be acquired with a second beam of radiation having a predetermined second angle different than the first angle. This is followed by determining axial fluid flow components from the first and second pluralities of measurements in both the first second data sets and calculating the fluid flow within the sample based on a combination of the determined axial fluid flow components and without using predetermined objective focal lengths for the first and second beams of radiation. Lastly the results of the calculation for fluid flow measurements may be output for storage or display.

The method may further include other processing elements, blocks, and/or steps. For example, determining a vessel cross sectional area for the first and second beams of radiation may be included. Determining an axial mean velocity for each data set over the vessel cross sectional area for the first and second beams of radiation may also be included. These actions may be followed by calculating a mean velocity ratio over the vessel cross sectional areas for the first and second data sets for the first and second beams of radiation using the determined axial velocity components and calculating the flow using the ratio of the first and second mean velocities for the first and second data sets, multiplying by the cross sectional areas, and dividing by an angle difference between the first and second beams of radiation.

In another aspect of the disclosure, the OCT system is a phase Doppler OCT system and the axial flow components are determined by calculating phase differences between the two or more measurements taken within a region substantially near a transverse location in first and second data sets.

In another aspect of the disclosure, the objective focal lengths of the first and second beams of radiation are the axial length of an eyeball.

In some aspects of the disclosure, the first and second data sets are acquired sequentially or simultaneously. The first and second data sets are acquired using one or more beams of radiation configured in a predetermined shape, such as concentric circular patterns.

In some aspects of the disclosure, the angle difference between the first angle and the second angle is chosen such that the signal-to-noise ratio of the phase shifts between the first and second beams of radiation are substantially similar. In some aspects of the disclosure the depth position of the first and second beams of radiation are substantially similar.

In another aspect of the disclosure, the present methods may be used for the diagnosis or treatment of a disease in a subject, the method comprising obtaining fOCT scans of a target, determining the flow of the bodily fluid from the fOCT scans generated in step (a), wherein the determining does not require an objective focal length and providing a medical decision.

In another aspect of the disclosure, the present methods may be used for an optical coherence tomography system configured to generate fOCT objective length free fluid flow measurements.

### III. General Methods for Objective Focal Length Free Flow Measurement

### A. Terminology of OCT Methods

The terms “optical coherence tomography” and “OCT,” described herein, generally refer to an interferometric technique for imaging samples, in some examples, with micrometer lateral resolution. This non-invasive optical tomographic imaging technique is used in a variety of medical and industrial applications to provide cross-sectional or 3D images of a target.

The terms “functional OCT” and “fOCT,” described herein, generally refer to a method of OCT imaging that provides for the acquisition of both structural (3D, tomographic and cross-sectional information) and functional information about a target, as described herein. In some examples, fOCT may refer to “visible-OCT” or “vis-OCT.” Vis-OCT generally refers to a type of fOCT that includes use of visible light. In some examples, OCT or fOCT may refer to OCT methods comprising use of near infrared (NIR) light.

As describe herein, fOCT may utilize any method of OCT. Generally, fOCT may be configured with an interferometer, as is the case for many other OCT methods. Light from a light source (for example, a broadband light source) is split (for example, by a beam-splitter) and travels along a sample arm (generally comprising the sample) and a reference arm (generally comprising a mirror). A portion of the light from the sample arm illuminates a target. Light is also reflected from a mirror in the reference arm. (Light from the test arm and the reference arm is recombined, for example, by the beam-splitter.) When the distance travelled by light in the sample arm is within a coherence length of the distance travelled by light in the reference arm, optical interference occurs, which affects the intensity of the recombined light. The intensity of the combined reflected light varies depending on the target properties. Thus, variations for the intensity of the reflectance measured are indications of the physical features or attributes of the target being imaged.

In some examples, the methods and systems of the disclosure may utilize time-domain OCT, where the length of the reference arm can be varied (for example, by moving one or more reference mirrors). The reflectance observed as the reference arm distance changes indicates sample properties at different depths of the sample. In some examples, the length of the sample arm is varied instead of or in addition to the variation of the reference arm length. In some examples, the devices, methods and systems may utilize frequency-domain OCT, where the distance of the reference arm can be fixed, and the reflectance can then be measured at different frequencies. For example, the frequency of light emitted from a light source can be scanned across a range of frequencies or a dispersive element, such as a grating, and a detector array may be used to separate and detect different wavelengths. Fourier analysis can convert the frequency-dependent reflectance properties to distance-dependent reflectance properties, thereby indicating sample properties at different sample depths. In certain examples, OCT can show additional information or data not obtainable from other forms of imaging.

In some examples, the methods and systems of the disclosure may utilize frequency-domain optical coherence tomography, where the reference and sample arms are fixed. Light from a broadband light source comprising a plurality of wavelengths is reflected from the sample and interfered with light reflected by the reference mirror/s. The optical spectrum of the reflected signal can be obtained. For example, the light may be input to a spectrometer or a spectrograph, comprising, for example, a grating and a detector array that detects the intensity of light at different frequencies.

In some examples, the methods and systems of the disclosure may utilize spectral domain optical coherence tomography, whereby spectral information is extracted by distributing different optical frequencies onto a detector stripe (for example, a line-array CCD or CMOS) via a dispersive element. Information of the full depth scan can be acquired within a single exposure.

Fourier analysis may be performed, for example, by a processor, and may convert data corresponding to a plurality of frequencies to that corresponding to a plurality of positions within the sample. Thus, data from a plurality of sample depths can be simultaneously collected without the need for scanning of the reference arm (or sample) arms. Additional details related to frequency domain optical coherence tomography are described in Vakhtin et al., (Vakhtin A B, Kane D J, Wood W R and Peterson K A. “Common-path interferometer for frequency-domain optical coherence tomography,” Applied Optics. 42(34), 6953-6958 (2003)) and incorporated by reference herein.

Other methods of performing optical coherence tomography are possible. For example, in some cases of frequency domain optical coherence tomography, the frequency of light emitted from a light source varies in time. Thus, differences in light intensity as a function of time relate to different light frequencies. When a spectrally time-varying light source is used, a detector may detect light intensity as a function of time to obtain optical spectrum of the interference signal. The Fourier transform of the optical spectrum may be employed as described herein. The devices, methods and systems of the disclosure may utilize any method of OCT, including but not limited to spectral domain OCT, Fourier domain OCT, time encoded frequency domain OCT, or swept source OCT, single point OCT, confocal OCT, parallel OCT, or full field OCT as known in the art.

Generally, the term “A-scan” OR “A-line” describes the light reflectivity associated with different sample depths. The term “B-scan” or “B-line” as used herein refers to the use of cross-sectional views of tissues formed by assembly of a plurality of A-scans. In the case of fOCT methods of cancer detection, light reflected by cancerous tissue target is converted into electrical signals and can be used to generate both cross-sectional or 3D structural images and metabolic functional information about the target tissue (such as cancerous growth, lesion, or tumor). In the case of ophthalmology, light reflected by eye tissues is converted into electrical signals and can be used to provide data regarding the 3D structure of tissue in the eye and metabolic activity in the retina. In many cases, including but not limited to cancer detection and ophthalmology, A-scans and B-scans can be used, for example, for differentiating normal and abnormal tissue.

For general methods, an A-scan can generally include collecting data at one or more transverse locations in a target, at a plurality of depths in a z-axis direction; a B-scan may include cross-sectional data from a medial border to a lateral border, or (x,y) axis direction. In the case of fOCT of a skin cancer lesion for example, an A-scan can generally include data from the outer regions of the epidermis of the lesion to the inner regions comprising vasculature, while B-scans can include cross sectional data from one lesion border to another in the (x,y) plane. In ophthalmic instances, an A-scan can generally include data from the cornea to the retina, and a B-scan can include cross-sectional data from a medial border to a lateral border of the eye and from the cornea to the retina. 3D C-scans may be used to generate one or more 3D images by combining a plurality of B-scans in variety of examples.

In the present disclosure, “target” may indicate any sample, object, or subject suitable for imaging. In some examples, a target may include but is not limited to inanimate material such as metals, alloys, polymers, and minerals as found for industrial applications for fOCT and as described herein. In some examples, a target may be animate material, such any suitable living material including but not limited to embryos, seeds, cells, tissues, grafts, blood vessels, organs, or organisms as would be suitable for medical and agricultural applications for fOCT as described herein. In some examples, a target may be retinal tissue, etc.

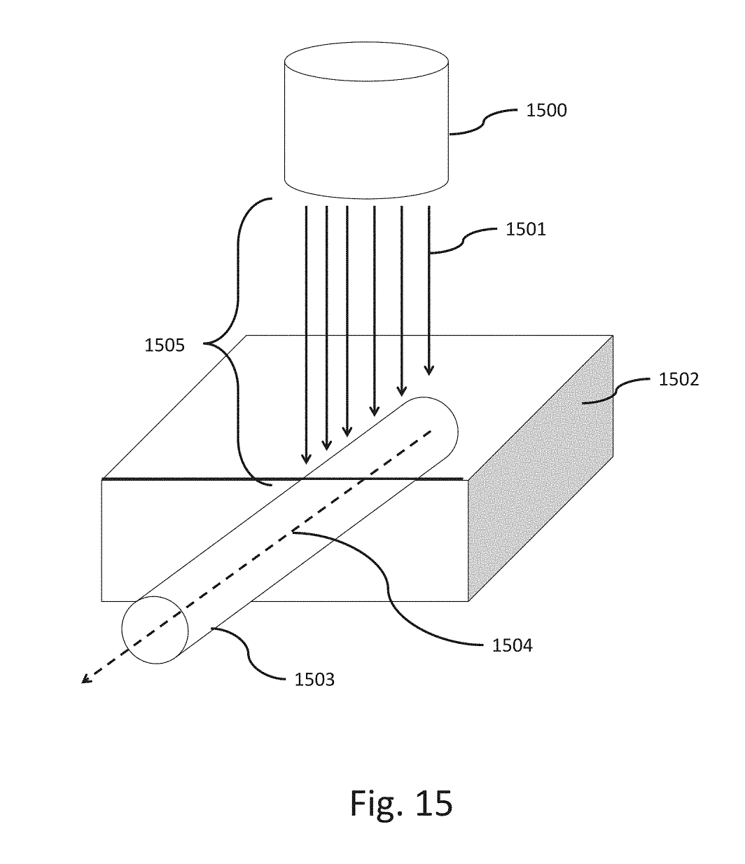

In the present disclosure, objective focal length may refer to any distance or length between the OCT objective and the target. As shown in FIG. 15, an OCT device or system 1500 may generate beams of radiation 1501 on a target 1502. The objective focal length 1505 may refer to a distance between 1500 and 1502. In some examples, the target may contain one or more vessels or blood vessels 1504, through which fluid 1503 flows at some velocity. The general methods and system of the present disclosure provide for the determination of fluid flow without relying on predetermining, measuring, or assuming the objective focal length 1505.

In some examples, where the target is the retina of an eyeball, the objective focal length may be the axial length of the eyeball as shown in FIG. 1. Light beams from an fOCT instrument enter the eyeball 100 through the outside of the cornea at V, and hit the retina at F′. In some examples, the axial length of the eye may be considered the distance from V to F′, or the sum of lengths of endpoints V and H′ 102 and endpoints H and F′, or l′ 101. In some examples, axial length may refer to the length l′ 101, the distance between endpoints H′ and F′.

In some cases, axial fluid flow components may refer to physical parameters relating to the movement of one or more particles in the fluid. For example, in blood, one or more blood components, such as blood cells may be imaged by Doppler OCT. axial fluid components of individual red blood cells in a blood vessel may include but are not limited to the blood vessel diameter, the velocity of the red blood cell and the Doppler angle of the imaging beam of radiation, as described herein.

### B. System Configurations for Objective Focal Length Free Flow Measurement

A fOCT system for data collection may be setup with a variety of configurations, and generally suitable with any type of OCT. FIG. 2a illustrates an example system 200 configured for objective focal length free flow measurements. The example free-space vis-OCT system 200 includes lens L1 202, lens L2 204, an x-y axis linear scanning mirror unit 206 (e.g., a pair of rotatable mirrors to steer the laser beam such as piezo-driven galvo mirrors (GM) or other rotation mechanisms such as resonance scanning mirrors, etc.), beam-splitter 208, dispersion control (DC) 210, reference mirror (REF) 212, laser 214 (e.g., generated by a supercontinuum source such as a continuous wave (CW) argon-ion laser, etc.), charge-coupled device (CCD) camera 216 (e.g., a two-dimensional CCD or other detector such as a CMOS camera, etc.), and computer or other processor 218.

Lenses L1 202 and L2 204 relay a beam generated by the laser 214 onto a target pupil 220. The beam-splitter 208 works with a reference arm including reference mirror 212 with dispersion control 210 to adjust the beam from the laser 214. The beam is directed by the mirrors 206 through the lenses 202, 204 to impact the pupil 220. Resulting image information is captured by the CCD 216 and relayed to the computer 218. The computer 218 can be used to control the CCD 216 and/or other components of the system 200, for example.

FIG. 2b illustrates an example illumination spectrum obtained using the system 200. FIG. 2c shows a comparison of theoretical and experimental axial resolutions obtained using the example system 200.

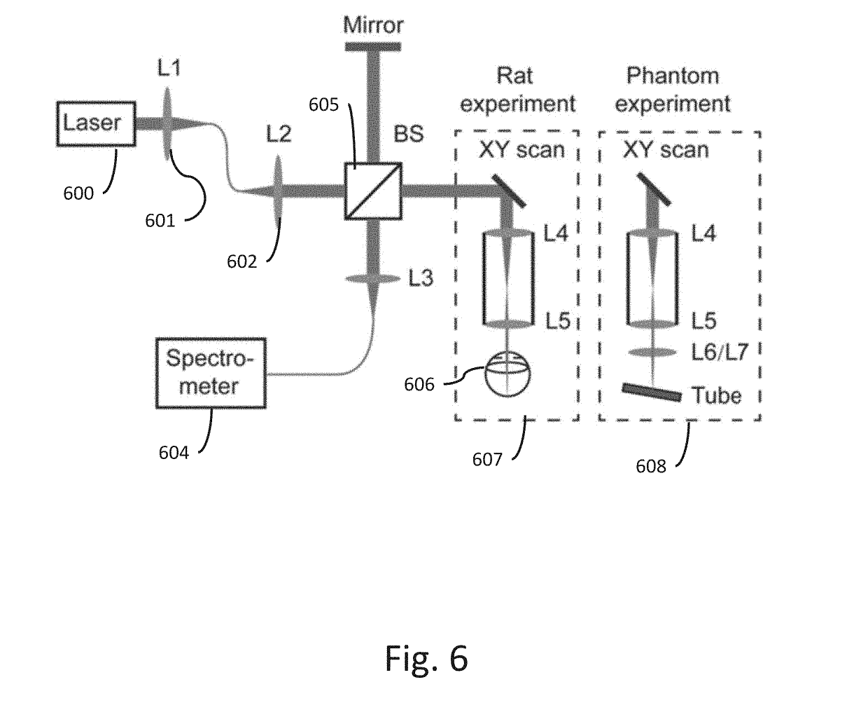

FIG. 6 provides another example of a fOCT configuration for objective focal free flow measurements. FIG. 6 provides a schematic of an example of a free-space SD-OCT where a supercontinuum laser source 600 (e.g., SuperK NKT photonics; center wavelength: 568.5 nm; bandwidth: 107 nm) may be used to generate one or more beams of radiation, which pass through lenses 601 and 602, are collimated onto a beam splitter 605, which splits the laser beam into two parts, one for the sample arm and one for the reference arm. In the sample arm, the laser beam may be scanned by a two-dimensional galvanometer (e.g., QS-7, Nutfield Technology) and relayed to through a telescope system to either an eye 606, for experiments in living subjects such as rats 607, or related to a phantom, such as a capillary tube 608, etc. The reflected photons from the sample arm and reference arm interfere with each other; then collected by a spectrometer 604.

In some examples, a supercontinuum source is used for illumination of a target. In some example, an open-space Michelson interferometry configuration may be adopted due to the minimum dispersion. The beam may also be collimated and split by a cube beam splitter into the reference and sample arms. The sample arm may include a two-dimensional galvo mirror to steer the beam, and, optionally, a 0.2 magnification Keplerian telescope to relay the beam from the galvo mirror to the target. The reference arm may include a dispersion control glass plate, and a mirror to illustrate the beam. The two beams from the reference and sample arms recombined at the beam splitter and may be collected by an optical fiber. The fiber may deliver the light to a spectrometer, which may include a collimating lens, a diffraction grating, an objective lens, and a line scan CCD camera (e.g., Balser, sprint slp2k). The camera exposure and the scanning galvo mirror may be synchronized by an analog output card.

The methods and systems, of the present disclosure may use any light source suitable for OCT, including but not limited to supercontinuum lasers, superluminescent diodes, continuous wave lasers or ultrashort pulsed lasers. The light source may be used to generate one or more low coherence beams of radiation or light to illuminate the target, for example.

In some examples, the light source may be used to generate one or more beams of light for objective focal length free flow measurements. In some examples, the number of beams used must be optimized. On one hand, as is known in the art, increasing the number of beams may increase the number of measurements made, which may improve the accuracy of flow measurements. However, as the number of beams used increases, the difficulty of aligning these beams may also increase. In some examples, an increase in the number of beams used may also increase the light or radiation exposure of the target. In ophthalmic applications, where the target tissue is the retina, minimizing exposure may be preferred. In some examples, a number of beams used may be 2 (a pair). In some examples, a number of beams may be 3. In some examples, the light source may be used to generate at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, and at least 20 beams of light. In some examples, the light source may be used to generate at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, at most 2 and at most 1 beams of light. In some examples, the light source may generate between 1 and 10 beams. In other examples, the light source may generate between 2 and 5 beams. In other examples, the light source may generate between 5 and 20 beams. In other examples, the light source may generate between 10-15 beams.

Generally, the wavelength range of the one or more beams of light may range from about 500 nm to about 620 nm. In some examples, the wavelength may range between 200 nm to 600 nm. In some examples, the wavelength may range between 300 to 900 nm. In some examples, the wavelength may range between 500 nm to 1200 nm. In some examples, the wavelength may range between 500 nm to 800 nm. In some examples, the wavelength range of the one or more beams of light may have wavelengths at or around 500 nm, 510 nm, 520 nm, 530 nm, 540 nm, 550 nm, 560 nm, 570 nm, 580 nm, 590 nm, 600 nm, 610 nm, and 620 nm. Generally, the wavelength range of the one or more beams of light may range from 200 nm to 800 nm. In some examples, the wavelength range of the one or more beams of light may have a wavelengths at or around 200 nm, 210 nm, 220 nm, 230 nm, 240 nm, 250 nm, 260 nm, 270 nm, 280 nm, 290 nm 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 900 nm, 1000 nm, 1100 nm, 1200 nm, 1300 nm, 1400 nm and 1500 nm. In some examples, fOCT and devices, methods, and systems of the present disclosure include two or more beams of light with wavelengths in the visible light spectrum or the near infrared (NIR) light spectrum. In some examples, fOCT includes beams of light with wavelengths in the visible light spectrum and the NIR spectrum.

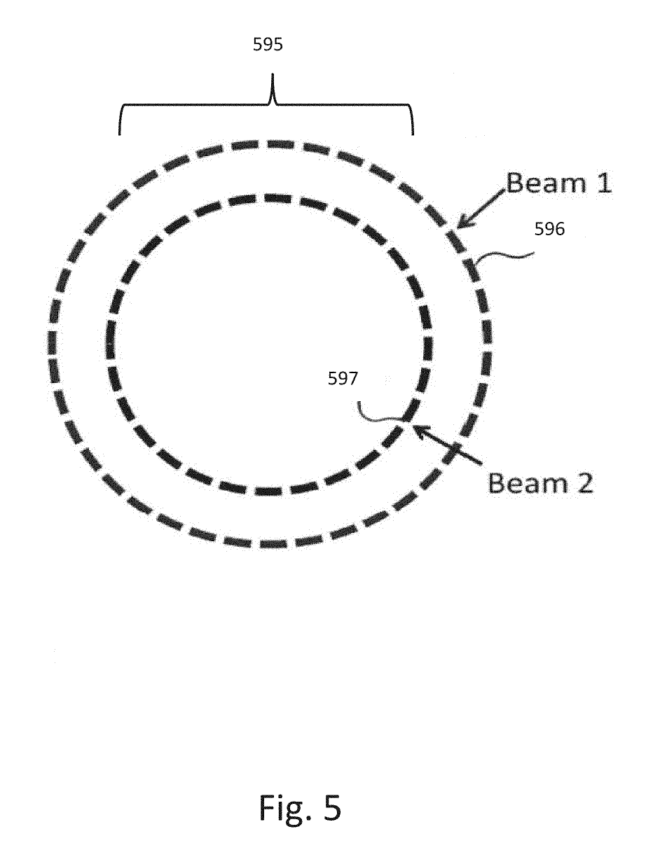

Generally, one or more beams of light used to illuminate a target may be configured in any suitable pattern. The pattern may be chosen based on the shape or pattern of the target, for example. In some examples, the beams of light may be one or more polygon patterns. In some examples, the illumination pattern may be a rectangle of one or more beams. In some examples, the beams of light may illuminate the target as one or more circles, or, shown in FIG. 5, two or more concentric circles 595, with one or more beams, 596, and 597. In some examples, a suitable pattern may be chosen based upon the pattern of vessels or fluid flow to be imaged in a target. For example, in a retina, blood vessels are found radially around the optic nerve head in a circular pattern or substantially circular pattern. For example, for fOCT imaging of a retina, one or more concentric circles of beams, or substantially circular beams, may be used to illuminate the target retina.

In some examples, where a pair of beams may be used, such as in FIG. 5, which shows a pair of beams of radiation configured as concentric circles, multiple pairs of beam radiation, configured as any pattern, may be used. Multiple pairs of beams of radiation may be used to average signal, or determine the consistency of axial flow measurements across one or more pairs of beams of radiation. In some examples, the number of pairs of beams used may be optimized and dependent on the nature of the fluid flow in the eye and the desired exposure of light radiation. For example, in imaging blood vessels in the retina, the pulsatile nature of the blood may require 5-10 pairs of radiation beams to measure a stable or consistent phase value. In some examples, 5-15 pairs of beams may be used. In some examples, a target may be exposed to at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, and at least 20, at least 30, and at least 40 pair(s) of beams of radiation. In some examples, the light source may be used to generate at most 40, at most 30, at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, at most 2 and at most 1 pair(s) of beams of radiation. In some examples, the light source may generate between 1 and 10 pair(s) of beams of radiation. In other examples, the light source may generate between 2 and 5 pair(s) of beams of radiation. In other examples, the light source may generate between 5 and 20 pair(s) of beams of radiation. In other examples, the light source may generate between 10-15 pair(s) of beams of radiation. In other examples, the light source may generate between 10-30 pair(s) of beams of radiation. In other examples, the light source may generate between 20-40 pair(s) of beams of radiation.

Further, the methods, and systems of the disclosure may allow for various power requirements to generate fOCT scans as compared to other OCT or imaging methods. In some examples, an fOCT device or system is configured to illuminate a target with a light source of at least 0.01 mW, at least 0.02 mW, at least 0.03 mW, at least 0.04 mW, at least 0.05 mW, at least 0.06 mW, at least 0.07, mW, at least 0.08 mW, at least 0.09 mW, at least 1.0 mW, at least 2.0 mW, at least 3.0 mW, at least 4.0 mW, at least 5.0 mW, at least 6.0 mW, at least 7.0 mW, at least 8.0 mW, at least 9.0 mW, at least 1.0 mW, at least 1.5 mW, at least 2.0 mW, at least 2.5 mW, at least 3.0 mW, at least 5.0 mW, at least 10.0 mW, at least 15.0 mW, and at least 20.0 mW. In some examples, an fOCT device or system is configured to illuminate a target with a light source of at most 0.01 mW, at most 0.02 mW, at most 0.03 mW, at most 0.04 mW, at most 0.05 mW, at most 0.06 mW, at most 0.07 mW, at most 0.08 mW, at most 0.09 mW, at most 1.0 mW, at most 2.0 mW, at most 3.0 mW, at most 4.0 mW, at most 5.0 mW, at most 6.0 mW, at most 7.0 mW, at most 8.0 mW, at most 9.0 mW, at most 1.0 mW at most 2.0 mW, at most 3.0 mW, at most 4.0 mW, at most 5.0 mW, at most 6.0 mW, at most 7.0 mW, at most 8.0 mW, at most 9.0 mW, at most 1.0 mW, at most 1.5 mW, at most 2.0 mW, at most 2.5 mW, at most 3.0 mW, at most 5.0 mW, at most 10.0 mW, at most 15.0 mW, and at most 20.0 mW. In some examples, a fOCT device or system is configured to illuminate a target with a light source of about 0.8 mW. In some examples, a fOCT device or system is configured to illuminate a target with a light source of about 0.5 mW-0.8 mW. In some examples, a fOCT device or system is configured to illuminate a target with a light source of about 0.1 mW-1.2 mW. In some examples, a fOCT device or system is configured to illuminate a target with a light source of about 0.2 mW-1.5 mW.

In some examples, the devices, methods, and systems of the disclosure allow for configuration of a fOCT device or system to acquire A-scans at a faster rate than other OCT or imaging methods. In some examples, A-scan acquisition rate may be at least 1 kHz, at least 2 kHz, at least 3 kHz, at least 4 kHz, at least 5 kHz, at least 6 kHz, at least 7 kHz, at least 8 kHz, at least 9 kHz, at least 10 kHz, at least 11 kHz, at least 12 kHz, at least 13 kHz, at least 14 kHz, at least 15 kHz, at least 16 kHz, at least 17 kHz, at least 18 kHz, at least 19 kHz, at least 20 kHz, at least 25 kHz, at least 30 kHz, at least 35 kHz, at least 40 kHz, at least 45 kHz, at least 50 kHz, at least 55 kHz, at least 60 kHz, at least 65 kHz, at least 70 kHz, at least 75 kHz, at least 80 kHz, at least 85 kHz, at least 90 kHz, at least 95 kHz, at least 100 kHz, at least 110 kHz, 120 kHz, at least 130 kHz, at least 140 kHz, at least 150 kHz, at least 160 kHz, at least 170 kHz, at least 180 kHz, at least 190 kHz, at least 200 kHz, at least 210 kHz, at least 220 kHz, at least 230 kHz, at least 240 kHz, and at least 250 kHz. In some examples, A-scan acquisition rate may be at most 1 kHz, at most 2 kHz, at most 3 kHz, at most 4 kHz, at most 5 kHz, at most 6 kHz, at most 7 kHz, at most 8 kHz, at most 9 kHz, at most 10 kHz, at most 11 kHz, at most 12 kHz, at most 13 kHz, at most 14 kHz, at most 15 kHz, at most 16 kHz, at most 17 kHz, at most 18 kHz, at most 19 kHz, at most 20 kHz, at most 25 kHz, at most 30 kHz, at most 35 kHz, at most 40 kHz, at most 45 kHz, at most 50 kHz, at most 55 kHz, at most 60 kHz, at most 65 kHz, at most 70 kHz, at most 75 kHz, at most 80 kHz, at most 85 kHz, at most 90 kHz, at most 95 kHz, at most 100 kHz, at most 110 kHz, at most 120 kHz, at most 130 kHz, at most 140 kHz, at most 150 kHz, at most 160 kHz, at most 170 kHz, at most 180 kHz, at most 190 kHz, at most 200 kHz, at most 210 kHz, at most 220 kHz, at most 230 kHz, at most 240 kHz, and at most 250 kHz. In some examples, A-scan acquisition rate may range from about 35 kHz to about 70 kHz. In some examples, A-scan acquisition rate may range from about 20 kHz to about 100 kHz. In some examples, A-scan acquisition rate may range from about 75 kHz to about 200 kHz. In some examples, A-scan acquisition rate may range from about 100 kHz to about 500 kHz.

Each B-scan may have a plurality of A-scans, ranging from 1 to 5000. In some examples, each B-scan may have at least about 1, 10, 20, 30, 40, 50, 60, 70, 80, 90, 100, 120, 140, 160, 180, 200, 210, 220, 230, 240, 250, 260, 270, 280, 290, 300, 400, 500, 600, 700, 800, 900, 1000, 1100, 1200, 1300, 1400, 1500, 1600, 1700, 1800, 1900. 2000, 2100, 2200, 2300, 2400, 2500, 2600, 2700, 2800, 2900, 3000, 4000, or 5000 A-scans. In some examples, each B-scan may have at most about 1, 10, 20, 30, 40, 50, 60, 70, 80, 90, 100, 120, 140, 160, 180, 200, 210, 220, 230, 240, 250, 260, 270, 280, 290, 300, 400, 500, 600, 700, 800, 900, 1000, 1100, 1200, 1300, 1400, 1500, 1600, 1700, 1800, 1900. 2000, 2100, 2200, 2300, 2400, 2500, 2600, 2700, 2800, 2900, 3000, 4000, or 5000 A-scans. In some examples, a B-scan may have 256 A-scans.

In some examples, fOCT may be performed such at least about 1, 100, 1000, 10000, 20000, 30000, 40000, 50000, 60000, 70000, 80000, 90000, 100000, 120000, 140000, 160000, 180000, 200000, 300000, 400000, 500000, 600000, 700000, 800000, 900000, and 1000000 A-scans are generated for quantitatively 3D-imaging in the target. In some examples, fOCT may be performed such at most about 1, 100, 1000, 10000, 20000, 30000, 40000, 50000, 60000, 70000, 80000, 90000, 100000, 120000, 140000, 160000, 180000, 200000, 300000, 400000, 500000, 600000, 700000, 800000, 900000, and 1000000 A-scans are generated for quantitatively 3D-imaging in the target. In some examples, about 65,000 A-scans are generated for quantitatively 3D-imaging in the target.

In some examples, an electrocardiogram (EKG) amplifier to collect an EKG signal may be used in addition to fOCT imaging. In some examples, an EKG signal may be useful when configuring a fOCT device or system for objective length free measurements of flow in a target. In some examples, these measurements may be related to metabolic imaging of blood vessels. The EKG signal collection may be synchronized with the scanning by an analog output card, so that the collections of EKG and the OCT image are simultaneous.

### C. Signal Processing Methods for Objective Focal Length Free Flow Measurement

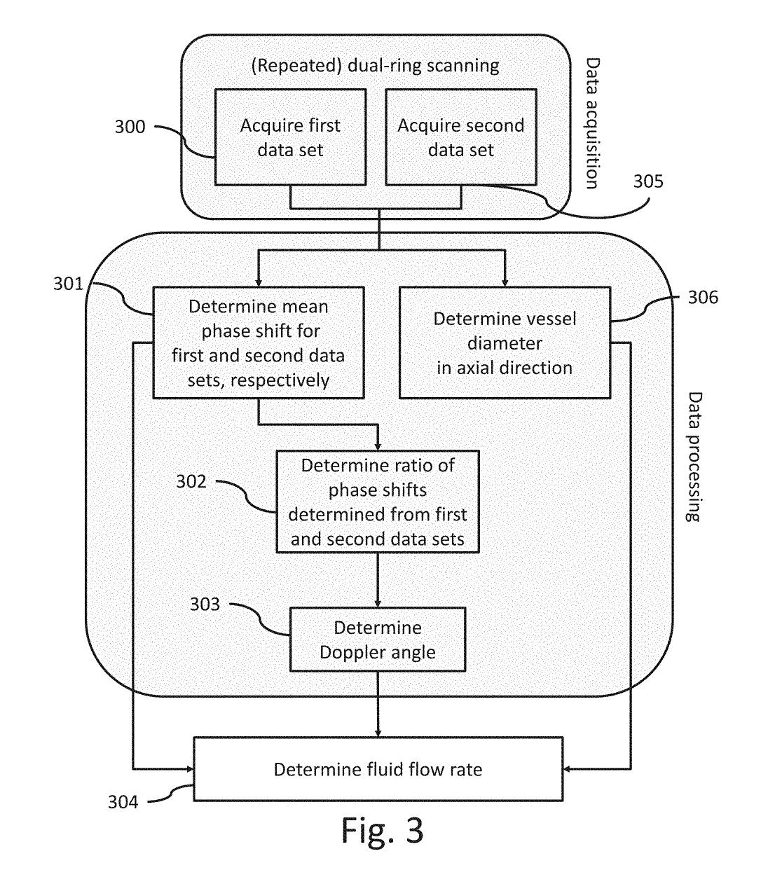

The present disclosure provides for general methods of processing OCT signal data for objective focal length free flow measurements. Generally, as shown in FIG. 3, first and second data sets, 300 and 305 respectively are acquired with an OCT device/system, such as those shown and described herein. Generally, a first OCT data set is acquired at one or more transverse locations in a target or subject using one or more OCT devices and/or systems. The first data set may include a first plurality of measurements, wherein at least two of the first plurality of measurements are made within a region substantially near the one or more transverse locations in the target using an OCT scanning device. The first data set is generally acquired with a first beam of light radiation from an OCT scanning device. In some examples, the beam of light may be configured as a specific shape, polygon, ring or circle. Next, a second data set is acquired for one or more transverse locations in a target or subject, wherein at least two measurements of the second plurality of measurements are made within a predetermined distance from the same transverse location as the at least two measurements of the first plurality of measurements. The second data set is generally acquired with a second beam of light radiation from an OCT scanning device. In some examples, the second beam of light may be configured as a certain shape, polygon, ring or circle which is concentric to the first beam of light.

After exposing the target to the one or more beams of light from the OCT device or system, scanning signal data is acquired by the OCT device or system Scanning data may be processed to extract the mean phase shift of the one or more beams of light using a software algorithm or computer which may be part of the OCT device or system. In some examples, the mean phase shift may be shifted as a result of flow in a fluid in the target or subject. This mean phase shift may be determined, 301. In some examples, the ratio of the phase shifts from the first and second data sets is determined, 302. In some examples, the OCT device or system, including a computer or software algorithm, may be used to determine the ratio of the phase shifts from the first and second data sets. The mean phase shift may allow for a Doppler angle to be determined, 303, in order to determine a flow velocity of a fluid in the subject or target. Additionally, the vessel diameter size may be determined, 306, by one or more vessel segmentation methods as known in the art. Vessel segmentation may be performed through the analysis of signal data obtained by the OCT device or system, including a computer or software algorithm, which may perform the analysis on one or more OCT scans. Together, after determining the mean phase shifts of the first and second data sets, the mean phase shift ratio of the first and second data sets, and the vessel diameter in the target, the fluid flow and rate may be determined, 304.

i. Calculation Methods for Objective Focal Length Free Flow Measurement

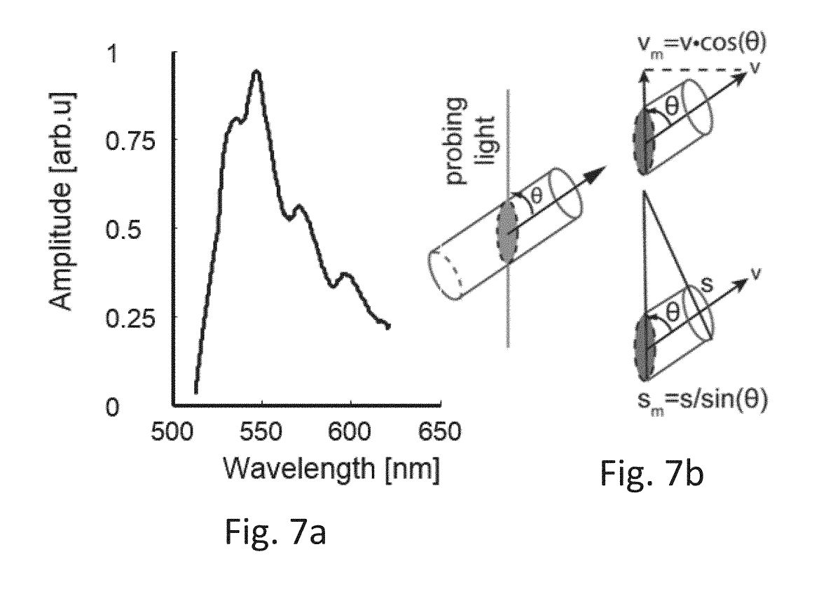

The calculation methods described herein may be performed by a software algorithm or computer of the OCT device/system. Generally, OCT scanning data is acquired by the OCT device or system and subsequently analyzed through the calculation methods described herein. The absolute flow rate F of the target can be expressed as any unit of distance divided by a time unit. In some examples, where the target sample is one or more retinal vessels in an eye, the absolute flow rate may be expressed as μl/min. Generally, axial flow components are a combination of absolute flow velocity V, which can be expressed as any suitable units of distance divided by time, (e.g. mm/s), and the perpendicular cross-sectional vessel size S of the vessel, (e.g. μm²). In some examples, the absolute flow rate F can be determined by multiplying the absolute flow velocity V by the perpendicular cross-sectional vessel size S of the vessel. Alternatively, as illustrated in FIG. 7b, F can also be quantified by the detected mean projected velocity Vₘ and the measured vessel area Sₘ from Doppler OCT as

$$F = VS = \frac{{V}_{m}}{cos ( θ )} {S}_{m} sin ( θ ) = {V}_{m} {S}_{m} tan ( θ ) , \qquad ( 1 )$$

where θ [degree] is the Doppler angle. Within Eq. 1, Vₘ can be calculated by

$${V}_{m} = \frac{{f}_{sample} {λ}_{0} Δφ}{4 π n} = \frac{{f}_{sample} {λ}_{0}}{4 π n} \frac{\int φ {dS}_{m}}{{S}_{m}} , \qquad ( 2 )$$

where fₛₐₘₚₗₑ [kHz] is the SD-OCT A-line rate; λ₀ [nm] is the center wavelength of the OCT (e.g. SD-OCT) light source spectrum as shown in FIG. 7a; and Δφ is the flow induced mean phase shift [rad] within the vessel.

The Sₘ can be measured within the cross-section B-scan as

$${S}_{m} = {π ( \frac{Dia}{2} )}^{2} = {π ( \frac{N\_P P\_L}{2} )}^{2} , \qquad ( 3 )$$

where Dia is the vessel diameter in the axial direction, N_P is the number of pixels in Dia, and P_L is the pixel size in the axial dimension of the image. N_P can be obtained from the B-scan image. P_L can vary. Generally, pixel size may vary with the type of spectrometer used. Pixel size in the axial dimension may be selected based on application. For example, pixel size may be chosen for applications requiring higher resolution, where imaged vessels are small. In applications where vessels are large, pixel size may be chosen for lower resolution requirements. In some examples, such as ophthalmic application, pixel size may be about 0.62 μm. In some examples, pixel size can be 0.1 μm to 1 μm. In some examples, pixel size is at least 0.1 0.2, 0.3, 0.4, 0.5 0.6, 0.7, 0.8, 0.9, 1, 2, 3, 4, or 5 μm. In some examples, pixel size is at most 0.1 0.2, 0.3, 0.4, 0.5 0.6, 0.7, 0.8, 0.9, 1, 2, 3, 4, or 5 μm. In some examples, P_L is from 0.5-1 μm. In some examples, P_L is from 1-5 μm. In some examples, P_L is from 0.3-1.5 μm. In some examples, P_L is from 0.6-3 μm.

Combining Eq. 1, Eq. 2 and Eq. 3, F can be calculated as

$$\begin{matrix}F = \frac{{f}_{sample} {λ}_{0} \int φ {dS}_{m}}{4 π n} tan ( θ ) \\ = \frac{{f}_{sample} {λ}_{0} {Δφπ ( \frac{N\_P P\_L}{2} )}^{2}}{4 π n} tan ( θ )\end{matrix} \qquad ( 4 )$$

According to Eq. 4, F can be extracted once the Doppler angle θ is known. If θ can be extracted free from the objective focal length, the flow rate of the fluid in the target can be measured without predetermining, or measuring this distance. For ophthalmic applications, the objective focal length may also be considered the eyeball axial length, and thus the retinal blood flow rate can be measured without knowing the eyeball axial length. In some examples, the objective focal length is equal to the axial length of an eyeball of a subject. In some examples, the objective focal length includes the axial length of an eyeball of a subject.

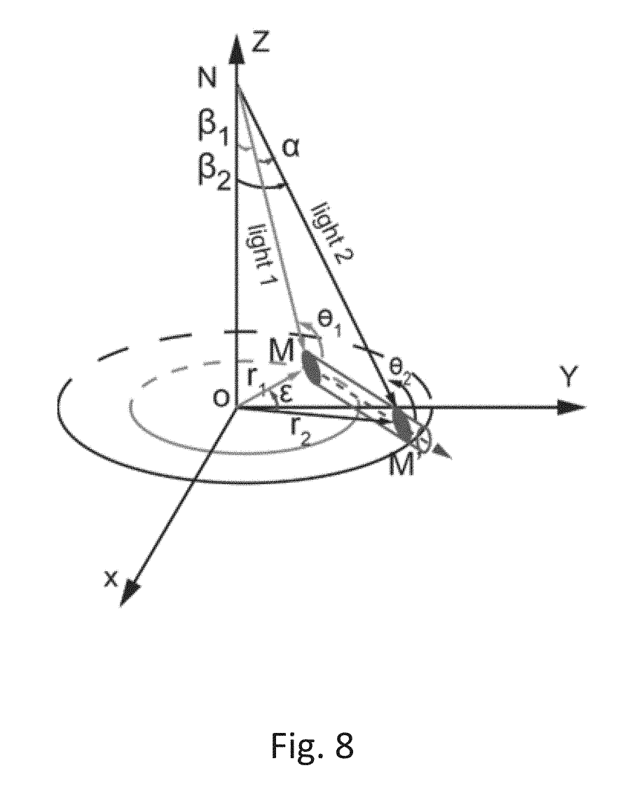

To assess the Doppler angle θ in an objective focal length (e.g. eyeball axial length) free manner, a dual-ring scanning protocol may be employed, where a pair of first and second beams of radiation are used. As shown in the schematic of FIG. 8, the geometry of the scanning configuration can be setup such that consecutively scanned pairs of concentric rings are exposed to a target (e.g., retina) with different preset angles β₁ and β₂. The first beam of radiation, β₁, may be set at first angle relative the sample and the second beam of radiation β₂, may be set a second angle relative to the second angle. The concentric scanning rings may intersect one sample vessel (e.g., retinal vessel) centerline at points M and M′, where the respective corresponding Doppler angles are θ₁ and θ₂, and the radius of the concentric scanning rings on the target (e.g., retina) are r₁ and r₂. The angle difference between β₁ and β₂ may be optimized, for example. In some examples, the angle difference between β₁ and β₂ is selected to be big enough to detect the phase difference between the two concentric rings. In some examples, the angle difference between β₁ and β₂ is set small enough such that the signal to noise ratio is substantially similar between the two concentric beam scans. In some example, scanning density may also be a factor. With smaller angles, a higher scanning density is required, which for some applications may be preferred. In some examples, the angle difference between β₁ and β₂ may be set small (e.g., 2 degrees) to ascertain the same or substantially similar depth locations of the concentric rings on the retina (e.g., for retinal applications, a 30 μm deviation in depth location). In some examples, the angle difference between β₁ and β₂ may be at least 0.2, 0.5, 0.75, 1, 2, 3, 4, 5, 6, 7, 8, 9, 10, 15, 20, 25, 30, 35, 40, 45, 50, 55, 60, 65, 70, 75, 80, 85, or 90 degrees. In some examples, the angle difference between β₁ and β₂ may be at most 0.2, 0.5, 0.75, 1, 2, 3, 4, 5, 6, 7, 8, 9, 10, 15, 20, 25, 30, 35, 40, 45, 50, 55, 60, 65, 70, 75, 80, 85, or 90 degrees. In some examples, the angle difference between β₁ and β₂ may be between 0.1-3 degrees. In some examples, the angle difference between β₁ and β₂ may be between 1-3 degrees. In some examples, the angle difference between β₁ and β₂ may be between 2-5 degrees. In some examples, the angle difference between β₁ and β₂ may be between 3-10 degrees. In some examples, the angle difference between β₁ and β₂ may be between 5-20 degrees. In some examples, the angle difference between β₁ and β₂ may be between 10-30 degrees. In some examples, the angle difference between β₁ and β₂ may be between 20-50 degrees. In some examples, the angle difference between β₁ and β₂ may be between 40-90 degrees.

In some examples, the same or substantially similar depth location may be dependent on the nature of the target to be imaged. For example, in retinal tissue, which may be about 250 um, it may be preferable to image the same or substantially similar depth location to image a single vessel, where the difference in depth between scans is 0 um (the same location). In some examples, the difference in depth may be substantially similar, ranging between 0-200 um and may depend on the size of the vessel(s) imaged. In some examples, the same or substantially similar depth locations between the first and second beams of radiation may be at least 0, 1, 5, 10, 15, 20, 25, 30, 35, 40, 45, 50, 75, 100, 125, 150, 175, or 200 um. In some examples, the same or substantially similar depth locations between the first and second beams of radiation may be at most 0, 1, 5, 10, 15, 20, 25, 30, 35, 40, 45, 50, 75, 100, 125, 150, 175, or 200 um. In some examples, the same or substantially similar depth locations between the first and second beams of radiation may be between 0 and 1 um. In some examples, the same or substantially similar depth locations between the first and second beams of radiation may be between 5 and 50 um. In some examples, the same or substantially similar depth locations between the first and second beams of radiation may be between 10 nm and 100 um. In some examples, the same or substantially similar depth locations between the first and second beams of radiation may be between 50 nm and 200 um.

In some examples, the angle difference between β₁ and β₂ may be set such that signal to noise ratio of the fOCT scan is at a suitable level. Signal to noise ratios may vary, depending on the type of OCT imaging used, or the type of OCT configuration. In some examples, the methods and systems of the disclosure may prefer imaging configurations that generate relatively high signal to noise ratio for the signal processing steps as described herein. In some examples, the angle difference between β₁ and β₂ may be set such that signal to noise ratio of one or more fOCT scans from one or more beams of radiations is at least 50, 55, 60, 65, 70, 75, 80, 85, 90, 95, 100, 120, 150 or 200 dB. In some examples, the angle difference between β₁ and β₂ may be set such that signal to noise ratio of one or more fOCT scans from one or more beams of radiations is at most 50, 55, 60, 65, 70, 75, 80, 85, 90, 95, 100, 120, 150, or 200. dB. In some examples, the angle difference between β₁ and β₂ may be set such that signal to noise ratio of one or more fOCT scans from one or more beams of radiations is between 50 and 75 dB. In some examples, the angle difference between β₁ and β₂ may be set such that signal to noise ratio of one or more fOCT scans from one or more beams of radiations is between 50 and 100 dB. In some examples, the angle difference between β₁ and β₂ may be set such that signal to noise ratio of one or more fOCT scans from one or more beams of radiations is between 75 and 120 dB. In some examples, the angle difference between β₁ and β₂ may be set such that signal to noise ratio of one or more fOCT scans from one or more beams of radiations is between 60 and 200 dB. In some examples, the angle difference between β₁ and β₂ may be set such that signal to noise ratio of one or more fOCT scans from one or more beams of radiations is between 75 and 125 dB.

In some examples, the angle difference between β₁ and β₂ may be set such that signal to noise ratio of the fOCT scan is the same or substantially similar. In some examples, setting the angle to achieve the same or substantially similar signal to noise ratio may help optimize a similar scanning density between the first and second beams, which may help improve the accuracy of objective focal length fluid measurements. In some examples, the angle difference between β₁ and β₂ may be set such that difference in signal to noise ratio of fOCT scans from is at least 0, 0.1, 0.5, 1, 2, 3, 4, 5, 6, 7, 8, 9, 10, 15, 20, or 25 dB. In some examples, the angle difference between β₁ and β₂ may be set such that difference in signal to noise ratio of fOCT scans from is at most 0, 0.1, 0.5, 1, 2, 3, 4, 5, 6, 7, 8, 9, 10, 15, 20, or 25 dB. In some examples, the angle difference between β₁ and β₂ may be set such that difference in signal to noise ratio of fOCT scans from is the same, where the signal to noise ratio difference is 0. In some examples, the angle difference between β₁ and β₂ may be set such that difference in signal to noise ratio of fOCT scans from is between 0.1 and 5 dB. In some examples, the angle difference between β₁ and β₂ may be set such that difference in signal to noise ratio of fOCT scans from is between 0.5 and 5 dB. In some examples, the angle difference between β₁ and β₂ may be set such that difference in signal to noise ratio of fOCT scans from is between 2 and 7 dB. In some examples, the angle difference between β₁ and β₂ may be set such that difference in signal to noise ratio of fOCT scans from is between 1 and 10 dB. In some examples, the angle difference between β₁ and β₂ may be set such that difference in signal to noise ratio of fOCT scans from is between 5 and 20 dB. In some examples, the angle difference between β₁ and β₂ may be set such that difference in signal to noise ratio of fOCT scans from is between 5 and 25 dB.

Assuming the absolute blood velocity within the sample vessel is V, then the corresponding velocity detected by probing beam NM′ and NM is:

Vₛ=V cos(θ₁), and (5)

V_{b}=V cos(θ₂)=V cos(θ₁+α), (6)

where α is the angle between the two probing beams. Combining Eq. 5 and 6:

$$\begin{matrix}\frac{{V}_{b}}{{V}_{s}} = \frac{V cos ( {θ}_{1} + a )}{V cos ( {θ}_{1} )} \\ = \frac{cos ( {θ}_{1} ) cos ( a ) - sin ( {θ}_{1} ) sin ( a )}{cos ( {θ}_{1} )} \\ = cos ( a ) - tan ( {θ}_{1} ) sin ( a ) .\end{matrix} \qquad ( 7 )$$

The Doppler angle can then be deducted from Eq. 7 as

$$tan ( {θ}_{1} ) = \frac{cos ( a ) - \frac{{V}_{b}}{{V}_{s}}}{sin ( a )} \qquad ( 8 )$$

From Eq. 8, the Doppler angle θ₁ can be obtained if the angle α is known. To obtain α, the objective focal length (e.g. eyeball axial length), (NO), is denoted as h, and the two probing beams' geometrical pathlengths (NM and NM′) are l₁ and l₂, respectively, as shown in FIG. 8. The lengths of r₁, r₂, l₁, and l₂ can be calculated by:

r₁=ₕ tan(φ₁),

r₂=h tan(φ₂),

l₁=h/cos(φ₁), and

l₂=h/cos(φ₂). (9)

Within the triangle M′OM, the length of M′M is denoted as l₃ and

l₃²=r₁²+r₂²−2r₁r₂ cos(ε), (10)

where the angle ε can be directly obtained from B-scan images from both rings of the concentric scanning rings. Within the triangle M′NM, the angle α can be calculated by

$$\begin{matrix}cos ( a ) = \frac{{l}_{1}^{2} + {l}_{2}^{2} - {l}_{3}^{2}}{2 {l}_{1} {l}_{2}} \\ = \frac{{h}^{2} {sec ( {ϕ}_{1} )}^{2} + {h}^{2} {sec ( {ϕ}_{2} )}^{2} - {h}^{2} {tan ( {ϕ}_{1} )}^{2} - {h}^{2} {tan ( {ϕ}_{2} )}^{2} + 2 {h}^{2} tan ( {ϕ}_{1} ) tan ( {ϕ}_{2} ) cos ( ɛ )}{2 {h}^{2} sec ( {ϕ}_{1} ) sec ( {ϕ}_{2} )} \\ = cos ( {ϕ}_{1} ) cos ( {ϕ}_{2} ) + sin ( {ϕ}_{1} ) sin ( {ϕ}_{2} ) cos ( ɛ ) .\end{matrix} \qquad ( 11 )$$

With calculated angle α, the Doppler angle θ can be obtained from Eq. 8, and the flow rate (e.g. as in retinal blood vessels) can be calculated further from Eq. 4. For ophthalmic applications, retinal blood flow is calculated without knowing the eyeball axial length.

In some examples, a first plurality of measurements taken near a transverse location with a first beam of radiation may be taken within a predetermined distance as measurements of the second plurality of measurements taken with a second beam of radiation. In some examples, the predetermined distance is at least 0, 0.001, 0.005, 0.1, 0.15, 0.2, 0.3, 0.4, 0.5, 0.6, 0.7, 0.8, 0.9, 1.0, 1.1, 1.2, 1.3, 1.4, 1.5, 1.6, 1.7 1.8, 1.9, 2.0, 3.0, 4.0, 5.0, 6.0, 7.0, 9.0, 10.0, 20, 30, 40, 50, 60, 70, 80, 90 or 100 μm. In some examples, the predetermined distance is at most 0, 0.001, 0.005, 0.1, 0.15, 0.2, 0.3, 0.4, 0.5, 0.6, 0.7, 0.8, 0.9, 1.0, 1.1, 1.2, 1.3, 1.4, 1.5, 1.6, 1.7 1.8, 1.9, 2.0, 3.0, 4.0, 5.0, 6.0, 7.0, 9.0, 10.0, 20, 30, 40, 50, 60, 70, 80, 90 or 100 μm. In some examples, the predetermined distance is between 0 and 0.5 μm. In some examples, the predetermined distance is between 0.1 and 1.0 μm. In some examples, the predetermined distance is between 0.5 and 2.0 μm. In some examples, the predetermined distance is between 1.0 and 5.0 μm. In some examples, the predetermined distance is between 1.5 and 10 μm. In some examples, the predetermined distance is between 0.5 and 50 μm. In some examples, the predetermined distance is between 10 and 90 μm. In some examples, the predetermined distance is between 20 and 100 μm.

ii. Vessel Segmentation for Accurate Objective Focal Length Free Flow Measurement

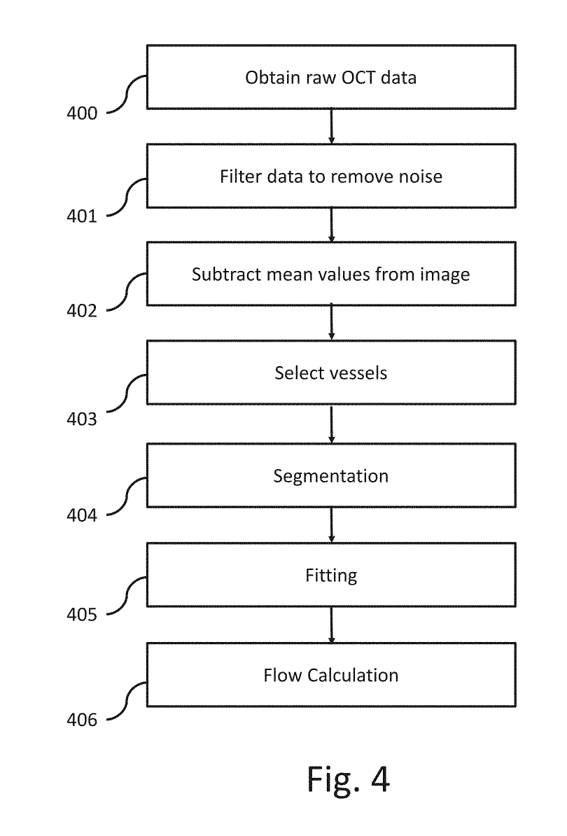

Accurate objective focal length free flow measurement using OCT or Doppler OCT may involve precise identification of the vessel location as well as vessel diameter from the bulk movements in retinal tissues. For ophthalmic applications, this may include the precise identification of retinal blood vessels. A simplified schematic of the general processing steps, elements, and/or blocks is shown in FIG. 4.

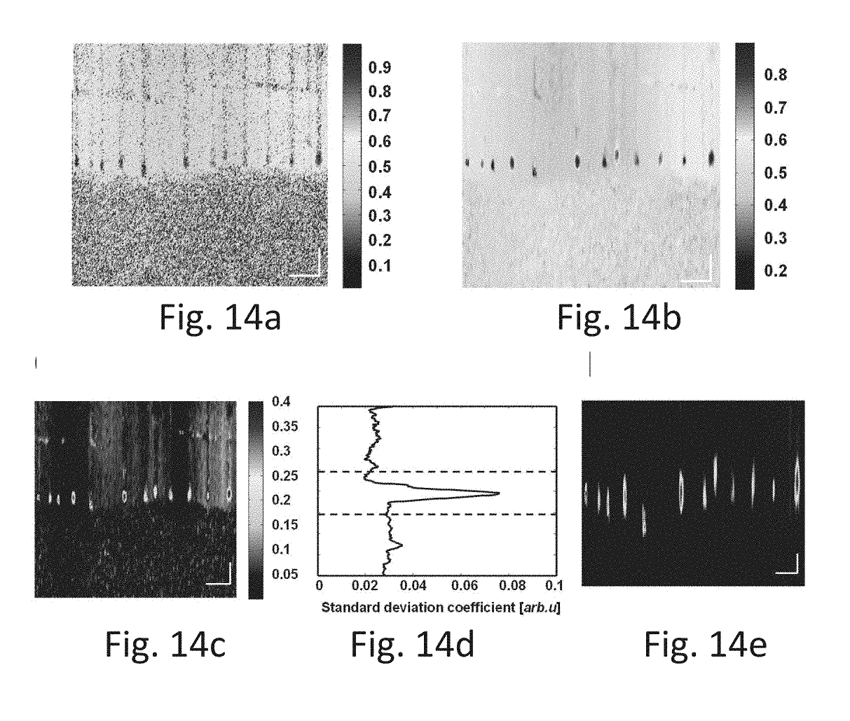

Raw OCT imaging data may first be obtained, as shown in FIG. 4, 400, such as from a SD-OCT system. In some examples, the OCT scanning data may be from retinal tissue. In some examples, where raw OCT data may be noisy a filter may be used to filter out the noise, 401. Any suitable filtering method, as practiced by one skilled in the art, may be used. In some examples, a median filter is used to filter noise. In some examples, Gaussian model filtering, Wiener filtering, or Wavelet filtering may be used. As shown in FIG. 14, a median filter is applied to raw data, FIG. 14a and removes the noise in the image, FIG. 14b. In some examples, segmentation and further reduce noise may be sped up during image and signal processing. Various methods to speed up this process may be used, including but not limited to subtracting the mean value of the image after filtering, as shown in FIG. 14c and as shown in FIG. 4, 402.

In some examples, vessels may be located in particular parts of the Doppler B-scan, as shown for retinal blood vessels in FIG. 14a as shown in FIG. 4, 403. In some examples, vessels may be identified and selected. Segmentation, as shown in FIG. 4, 404 may be performed only on the selected regions thought to contain vessels. To select regions for segmentation, various methods may be employed, including but not limited to calculating the standard deviation of the image horizontally, as shown in FIG. 14d. Regions containing a vessel may demonstrate greater standard deviation than regions not containing a vessel. Selection of the region may be based on numerous factors including but not limited to the relative magnitude of the standard deviation of the image signal across the image. As shown in FIG. 14, a region with more standard deviation from the rest of the image (as delineated with dashed lines in FIG. 14e), may be selected for further image processing steps for objective length free flow measurements.

In some examples, other methods for filtering and segmentation may be used. In some examples, a histogram based methods may be used. However, with data containing a high signal to noise ratio, other methods may be used as known by one skilled in the art. For example, support vector machine, in situ adaptive tabulation, kernel machines, Fisher kernals, platt scaling, polynomial kernals, relevance vector machine, sequential minimal optimization, active contour method, Winnow algorithms, and various other methods related to predictive analytics may be used for filtering and segmentation. In some examples, artificial neural networks, Fuzzy C-meaning clustering, or maximum likelihood estimation may be used for segmentation methods. In some examples, the active contour method may be used and may be preferable where high tolerance of noise is needed or poor signal to noise in the image still may yield accurate segmentation. In other examples, a method may be chosen to minimize or otherwise reduce time for processing such that image filtering and segmentation can be done in real time, or near the time of one or more scans.

With respect to utilizing the active contour method (ACM) for an objective focal length free flow measurement, for an given image I(x,y) in domain Ω, the ACM is formulated by minimizing or otherwise reducing the following energy equation:

$$E = {λ}_{1} {\int }_{inside ( c )} {\vert I ( x , y ) - {c}_{1} \vert}^{2} dxdy + {\int }_{outside ( c )} {\vert I ( x , y ) - {c}_{2} \vert}^{2} dxdy \qquad ( 12 )$$

where C is the contour, and C₁ and C₂ are the average intensity inside and outside of the contour respectively. The Eq. 12 can be solved iteratively: the whole image can initially be treated as the contour, dimension of the contour can be reduced and the corresponding C₁ and C₂ for each contour estimated iteratively. When differences of C₁ and C₂ between sequential contours are very small (like less than 10⁻⁵), segmentation may be assumed to be finished.

In some examples, at least 1, 2, 3, 4 5, 6, 7, 8 9, 10, 20, 30, 40, 50 100, 150, 200, 250, 300, 350, 400, 450, 500 or 1000 iteration(s) may be performed. In some examples, at most 1, 2, 3, 4 5, 6, 7, 8 9, 10, 20, 30, 40, 50 100, 150, 200, 250, 300, 350, 400, 450, 500 or 1000 iteration(s) may be performed. In some examples, 1-10 iterations are performed. In some examples, 5-20 iterations are performed. 7-50 iterations are performed. 30-100 iterations are performed. 100-250 iterations are performed. 250-500 iterations are performed.

For simplicity of solving Eq. 12 iteratively, in each iteration it can be assumed that:

$$\{ \begin{matrix}C = \{ ( x , y ) ∈ Ω , φ ( x , y ) = 0 \} , \\ inside ( C ) = \{ ( x , y ) ∈ Ω , φ ( x , y ) > 0 \} , \\ outside ( C ) = \{ ( x , y ) ∈ Ω , φ ( x , y ) < 0 \} ;\end{matrix} \qquad ( 13 )$$

By reducing or minimizing the energy in Eq. 12, the C₁ and C₂ can be solved as follows:

$$\begin{matrix}{c}_{1} ( φ ) = \frac{{\int }_{Ω} I ( x , y ) H ( φ ) dxdy}{{\int }_{Ω} H ( φ ) dxdy} & ( 14 ) \\ {c}_{2} ( φ ) = \frac{{\int }_{Ω} I ( x , y ) \cdot ( 1 - H ( φ ) ) dxdy}{{\int }_{Ω} ( 1 - H ( φ ) ) dxdy} & ( 15 ) \\ where H ( φ ) = \{ \begin{matrix}1 , φ \geq 0 \\ 0 , otherwise\end{matrix} . & \end{matrix}$$

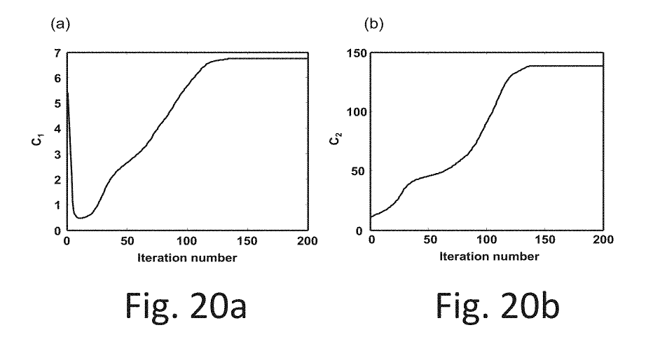

For each iteration, the value of ϕ(x, y) will be changed, and ϕ(x, y) is a function of time. The variation of ϕ(x, y) can be estimated as follows:

$$\frac{\partial φ}{\partial t} = \frac{I ( x , y ) - \frac{{c}_{1} ( φ ) + {c}_{2} ( φ )}{2}}{max ( \vert I ( x , y ) - \frac{{c}_{1} ( φ ) + {c}_{2} ( φ )}{2} \vert )} \cdot α \vert ∇ φ \vert , \qquad ( 16 )$$

where α is a factor added to increase the segmentation speed, and ∇(⋅) is the function for estimating the gradient. Example results of this method, showing C₁ and C₂ are reflected in FIG. 20a and FIG. 20b respectively. After more than 150 iterations, values of C₁ and C₂ may be constant, which may indicate segmentation can be finished.

The mean phase shifts ratio of the concentric rings may be considered stable or substantially similar to a point that segmentation can be considered finished by setting various thresholds. In some examples, the mean phase shifts ratio of the concentric rings may be considered stable or substantially similar between the adjacent cumulative B-scans when there was difference less than at least 0, 0.001, 0.002, 0.003, 0.004, 0.005, 0.006, 0.007, 0.008, 0.009, 0.1 rad. In some examples, the mean phase shifts ratio of the concentric rings may be considered stable or substantially similar between the adjacent cumulative B-scans when there was difference less than at most 0, 0.001, 0.002, 0.003, 0.004, 0.005, 0.006, 0.007, 0.008, 0.009, 0.1 rad. In some examples, the mean phase shifts ratio of the concentric rings may be considered stable or substantially similar between the adjacent cumulative B-scans when there was difference between 0.001 and 0.005 rad. In some examples, the mean phase shifts ratio of the concentric rings may be considered stable or substantially similar between the adjacent cumulative B-scans when there was difference between 0.002 and 0.008 rad. In some examples, the mean phase shifts ratio of the concentric rings may be considered stable or substantially similar between the adjacent cumulative B-scans when there was difference between 0.004 and 0.1 rad.

After segmentation, segmented vessels may also be fitted to generate more accurate results, as shown in FIG. 4, 405. Segmented vessels may be fitted with any suitable shape or pattern and may include but are not limited to circles, circular shapes, ellipses, rectangles, squares, triangles or any type of regular or irregular shaped polygon. In some examples, blood vessels may be ellipse fitted to generate more accurate results for fluid flow determinations, as shown in FIG. 4, 406.

### D. Objective Focal Length Free Flow Measurements of Analytes in a Target

The present disclosure provides for objective focal length free flow measurement of a fluid. In some examples, fluids may contain analytes. Analytes may refer to any living, chemical or biochemical moiety suitable for imaging. In some examples, this may include but is not limited to cells, oxygen, hemoglobin, oxygenated hemoglobin, deoxygenated hemoglobin, glucose, sugar, blood area nitrogen, lactate, hematocrit, biomarker, molecular marker, or nucleic acid that maybe suitable to image to determine target function. The analytes may also be molecules, including but not limited to: polypeptides, proteins, antibodies, enzymes, nucleic acids, saccharides, small molecules, drugs, and the like.

In some examples, the methods, and systems of the disclosure provide for “label-free” objective focal length free flow measurements in a target. In these examples, flow is measured without the use of exogenous reagents contacted with either the analytes or the targets. For example, a variety of imaging methods have been described describing quantifying flow with the use of contrast reagents or additional chemical markers or signals that may bind to one more analytes. The methods, and systems of the disclosure provide an imaging system where flow measurements are made label-free.

In some examples, however, one or more contrast reagents may be used in conjunction with the methods, and systems of the disclosure. In these examples, objective focal length free flow measurements may be obtained without a label, while one more flow measurements may be determined by contacting one or more analytes with an exogenous reagent such as contrast reagent.

A target may include any vessel or structure that can contain a fluid to be imaged including but not limited to tissue, healthy tissue, diseased tissue, retina, tumor, cancer, growth, fibroid, lesion, skin, mucosal lining, organ, graft, blood supply and one or more blood vessels.

In some examples, a fluid may be any material capable of flow, in which there may be particles that may be imaged by OCT or Doppler OCT. Bodily fluid may include but is not limited to whole blood, blood plasma, blood serum, urine, semen, tears, sweat, saliva, lymph fluid, pleural effusion, peritoneal fluid, meningal fluid, amniotic fluid, glandular fluid, spinal fluid, conjunctival fluid, vitreous, aqueous, vaginal fluid, bile, mucus, sputum and cerebrospinal fluid.

### IV. Functional OCT and Metabolic Activity

A. fOCT Objective Focal Length Free Flow Measurements and Target Function

In some examples, target function may include but is not limited to metabolic activity, metabolic rate, oxygen consumption, tissue consumption of a biomarker or analyte, pathophysiological alterations, pathological alterations, histological change such as tissue remodeling, abnormal growth of one or more blood vessels, or abnormal tissue growth, necrosis, apoptosis, necrosis, angiogenesis, cell proliferation, neurmodulation, neural activity, wound healing, infection, burns, scarring, radiological damage, hypoxia, oxidative stress and the like.

In some examples, measurements regarding flow rate of fluid such as blood may be used to compute or determine target function. For example, measurements regarding the flow rate of blood may help determine the flow rate of oxygen (via hemoglobin transport) into or out of a particular target or region. The flow of oxygen may be a critical factor in determining metabolic activity, histological change such as tissue remodeling, abnormal growth of one or more blood vessels, or abnormal tissue growth, necrosis, apoptosis, necrosis, angiogenesis. In other examples, the measurements of flow of other analytes or cells in fluids such as cerebrospinal fluid (CSF), may indicate the presence of disease of infection or inflammation of one or more parts of the nervous system.

In some example, a change in target function may be determined by comparing information from objective focal length free flow measurement of a fluid to a reference. In some examples, a reference many include but is not limited to measurements of from a healthy or normal target, one or more previous measurements, or an average of information from healthy subjects. In some examples, a reference may include objective focal length free flow measurement at different times. In some examples, one or more references may be compared to other references to determine a change in flow measurements.

### V. Medical Applications

In various examples, one or more fOCT images may provide objective focal length free flow measurement data from which a diagnosis and/or evaluation may be made. In some examples, such determinations may relate to biologic tissue structure, vasculature, and/or microcirculation. In some examples, objective focal length free flow measurements of blood flow through individual vessels therein may be useful in understanding mechanisms behind a number of disease developments and treatments including, for example, ischemia, degeneration, trauma, seizures, and various other neurological diseases. In still other examples, an fOCT objective focal length free flow measurement data image and techniques herein disclosed may be used to identify cancer, tumors, dementia, and ophthalmologic diseases/conditions (including, e.g., glaucoma, diabetic retinopathy, age-related macular degeneration). Still further, in various examples, OCT techniques as herein disclosed may be used for endoscopic imaging or other internal medicine applications. In some examples, fOCT objective focal length free flow measurement data may be used to stratify treatment options, such as personalizing or tailoring a patient treatment specific treatment protocol. In some examples, fOCT objective focal length free flow measurement data may be used for companion diagnostic decisions for the administration of one or more drugs. In some examples, fOCT objective focal length free flow measurement data may also be used to assess efficacy of a drug treatment during monitoring of a disease. In some examples, fOCT objective focal length free flow measurement data may also be used to screen drug efficacy during drug development. The foregoing examples of diagnosis and/or evaluation are provided for purposes of illustration only, and, thus, embodiments of the present invention are not limited to the examples disclosed and described herein.

A. fOCT Objective Focal Length Free Flow Measurement Data and Medical Decisions

In some examples, fOCT may be used to provide a medical decision. In some examples, a medical decision may include but is not limited to a treatment step, diagnostics, monitoring, follow-up, evaluation, confirmation of a diagnosis, prognosis, selecting a drug for a patient, changing a drug treatment to another drug, stopping a drug treatment, changing a treatment or drug dosage, increasing or decreasing frequency of treatment or drug administration, or recommending further evaluation. In some examples, a medical decision may be the guidance of a surgical tool or a surgical operation. In some examples, a medical decision may be the placement of one or more medical instruments or tools, such as the placement of a stent, or the placement of a suture. In some examples, a medical decision may be the determining of surgical margins in the excision of a tumor.

### B. Molecular Markers and Bodily Fluids

In some examples, fOCT objective focal length free flow measurement data may be used to detect or quantify a variety of molecular markers, which may be associated with a disease. The term molecular marker as defined herein includes, but is not limited to, a molecule or biomolecule, a whole cell, red blood cells or a commercially important substrate that may need to be tracked for its flow rate, distribution or identification. Molecules and biomolecules include nucleic acids, peptides, proteins, oligosaccharides, lipids, antigens, and small molecules. Commercially important substrates include, but are not limited to, organic and inorganic polymers, small molecules or chemical moieties or products made therefrom. In some examples, the molecular marker may include but is not limited to oxygen, hemoglobin, oxygenated hemoglobin, deoxygenated hemoglobin, glucose, sugar, blood area nitrogen, lactate, hematocrit, biomarker and nucleic acid.

In some examples, the concentration of one or more molecular markers may be determined in one or more bodily fluids. Generally, any bodily fluid may be suitable for imaging with fOCT. In some examples, a bodily fluid may include but is not limited to whole blood, blood plasma, blood serum, urine, semen, tears, sweat, saliva, lymph fluid, pleural effusion, peritoneal fluid, meningal fluid, amniotic fluid, glandular fluid, spinal fluid, conjunctival fluid, vitreous, aqueous, vaginal fluid, bile, mucus, sputum and cerebrospinal fluid.

### C. Stratification of Treatment Decisions

The methods and systems of the present disclosure can include using the status of one or more molecular markers determined in a target to stratify (rank) treatment options for a subject. In some examples, treatment may include any medical decisions as described herein. In some examples, one or more drugs may be stratified based on information determined by fOCT objective focal length free flow measurement data. The stratifying of drug treatments can be based on scientific information regarding the molecular markers. For example, the scientific information can be data from one or more studies published in one or more scientific journals (e.g., New England Journal of Medicine (NEJM), Lancet, etc.). The scientific information can be data provided in a commercial database (e.g., data stored in a database provided by Ingenuity® Systems). One or more pieces of scientific information can be used to stratify the treatments. In some examples, the data or scientific information may not be published. In some examples, the data or scientific information is maintained in a private database and used for comparison across select patients or sub groups of patients.

i. Classes of Drugs

Drug treatment options can be stratified into classes based on the status of one or more molecular markers in a target. For example, a first class of drug treatment options can be those for which scientific information predicts a drug will be efficacious for a subject whose target has one or more molecular markers of a particular status. Drugs in this first class can be a recommended drug treatment option for a subject.

A second class of drug treatment options can be those for which some scientific information predicts a drug will be efficacious for a subject with one or more molecular markers of a particular status, and some scientific information does not support use of the drug for the subject, based on one or more molecular markers of a particular status in a sample from a subject. For example, a sample may contain a marker whose status indicates the drug will be efficacious in the subject and another marker (e.g., a particular metabolic or fluid flow profile that indicates a specific disease state or stage) or may indicate the drug would also have a toxic effect on the subject.

This second class can also include drugs for which there is indirect scientific support for drug efficacy in a subject (e.g., the drug targets a protein that is in the same molecular pathway as a molecular marker in a target). For example, a drug in this class could target a kinase that functions downstream of an overexpressed variant of vascular endothelial growth factor (VEGF), which correlates with higher metabolic rate in a target as determined by fOCT. A drug in this second class can be a recommended drug treatment option for a subject.

A third class of drugs can be those for which scientific information indicates the drug will not be efficacious in the subject based on the status of one or more molecular markers in a sample from the subject. For example, a drug that targets a cell surface receptor may not display efficacy if information provided by fOCT imaging does not correlate well if efficacy. It can be recommended that a subject not be treated with a drug in this third class.

The drug treatment options can be stratified using an algorithm-based approach. The status of one or more molecular markers in a patient sample is determined. The scientific literature or a database of curated fOCT scans of one or more similar targets of one or more subjects is analyzed for information related to the status of the molecular marker and the efficacy of one or more different drugs. If the status of a molecular marker correlates with efficacy of a drug, then a recommendation can be made to treat the subject with that drug. If the status of a molecular marker does not correlate with efficacy of a drug, then a recommendation can be made not to treat a subject with the drug. A computer and computer readable medium can be used to stratify the drug treatment options.

A list of stratified drug treatment options can be presented in the form of a report. The stratification of drug treatment options can be indicated by color coding. For example, drugs in the first class can be color coded in green, drugs in the second class can be color coded in yellow, and drugs in the third class can be color coded in red.

The recommendation of a drug treatment option for a subject can be based on the stage of the diseases, (e.g. cancer of the subject, e.g., a late stage cancer, AMD, late stage AMD). Drug treatment options can also be stratified based on other factors, e.g., the type of disease, age of the subject, status of drug metabolism genes (genes involved in absorption, distribution, metabolism, and excretion), efficacy of other drugs the patient has received, clinical information regarding the subject, and family medical history.

In some examples, particular classes of drugs may be useful for treatment. In some examples, when fOCT objective focal length free flow measurement data is used to determine metabolic rate of tissues as a result of abnormal blood vessel proliferation or decrease, administration of drugs known to affect blood vasculature may be suitable. In some examples, this may include but is not limited to an angiogenesis inhibitor, e.g., a VEGF pathway inhibitor, e.g., a VEGF pathway inhibitor described herein, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor.

### D. Diseases

fOCT objective focal length free flow measurement data may be used in medical decisions related to a variety of diseases. These may include neurological diseases, which may include but is not limited to dementia, concussion, Alzheimer's disease, Parkinson's disease, peripheral neuropathy, epilepsy and multiple sclerosis. In some examples, these may include vascular diseases, including but not limited to diabetes, peripheral vascular diseases, stroke, cardiovascular diseases, myocardial infarction, and aneurysm.

In some examples, fOCT objective focal length free flow measurement data may be used to provide medical decision for ocular diseases which may include but is not limited to autosomal retinitis pigmentosa, autosomal dominant retinitis punctual albescens, butterfly-shaped pigment dystrophy of the fovea, adult vitelliform macular dystrophy, Norrie's disease, blue cone monochromasy, choroideremia, gyrate atrophy, age-related macular degeneration, retinoblastoma, anterior and posterior uveitis, retinovascular diseases, cataracts, corneal dystrophies, retinal detachment, degeneration and atrophy of the iris, and diabetic retinopathy, herpes simplex virus infection, cytomegalovirus, allergic conjunctivitis, dry eye, lysosomal storage diseases, glycogen storage diseases, disorders of collagen, disorders of glycosaminoglycans and proteoglycans, sphinogolipodoses, mucolipidoses, disorders of amino acid metabolism, dysthyroid eye diseases, anterior and posterior corneal dystrophies, retinal photoreceptor disorders, corneal ulceration, glaucoma and ocular wounds.

In some examples, fOCT objective focal length free flow measurement data may be used for medical decisions related to cancer, for example, acute myeloid leukemia; bladder cancer, including upper tract tumors and urothelial carcinoma of the prostate; bone cancer, including chondrosarcoma, Ewing's sarcoma, and osteosarcoma; breast cancer, including noninvasive, invasive, phyllodes tumor, Paget's disease, and breast cancer during pregnancy; central nervous system cancers, adult low-grade infiltrative supratentorial astrocytoma/oligodendroglioma, adult intracranial ependymoma, anaplastic astrocytoma/anaplastic oligodendroglioma/glioblastoma multiforme, limited (1-3) metastatic lesions, multiple (>3) metastatic lesions, carcinomatous lymphomatous meningitis, nonimmunosuppressed primary CNS lymphoma, and metastatic spine tumors; cervical cancer; chronic myelogenous leukemia (CML); colon cancer, rectal cancer, anal carcinoma; esophageal cancer; gastric (stomach) cancer; head and neck cancers, including ethmoid sinus tumors, maxillary sinus tumors, salivary gland tumors, cancer of the lip, cancer of the oral cavity, cancer of the oropharynx, cancer of the hypopharynx, occult primary, cancer of the glottic larynx, cancer of the supraglottic larynx, cancer of the nasopharynx, and advanced head and neck cancer; hepatobiliary cancers, including hepatocellular carcinoma, gallbladder cancer, intrahepatic cholangiocarcinoma, and extrahepatic cholangiocarcinoma; Hodgkin disease/lymphoma; kidney cancer; melanoma; multiple myeloma, systemic light chain amyloidosis, Waldenstrom's macroglobulinemia; myelodysplastic syndromes; neuroendocrine tumors, including multiple endocrine neoplasia, type 1, multiple endocrine neoplasia, type 2, carcinoid tumors, islet cell tumors, pheochromocytoma, poorly differentiated/small cell/atypical lung carcinoids; Non-Hodgkin's Lymphomas, including chronic lymphocytic leukemia/small lymphocytic lymphoma, follicular lymphoma, marginal zone lymphoma, mantle cell lymphoma, diffuse large B-Cell lymphoma, Burkitt's lymphoma, lymphoblastic lymphoma, AIDS-Related B-Cell lymphoma, peripheral T-Cell lymphoma, and mycosis fungoides/Sezary Syndrome; non-melanoma skin cancers, including basal and squamous cell skin cancers, dermatofibrosarcoma protuberans, Merkel cell carcinoma; non-small cell lung cancer (NSCLC), including thymic malignancies; occult primary; ovarian cancer, including epithelial ovarian cancer, borderline epithelial ovarian cancer (Low Malignant Potential), and less common ovarian histologies; pancreatic adenocarcinoma; prostate cancer; small cell lung cancer and lung neuroendocrine tumors; soft tissue sarcoma, including soft-tissue extremity, retroperitoneal, intra-abdominal sarcoma, and desmoid; testicular cancer; thymic malignancies, including thyroid carcinoma, nodule evaluation, papillary carcinoma, follicular carcinoma, Hürthle cell neoplasm, medullary carcinoma, and anaplastic carcinoma; uterine neoplasms, including endometrial cancer and uterine sarcoma.

In some examples, a medical decision may be facilitated by comparing the bodily fluid flow in a target (e.g. tissue), by comparing the bodily fluid flow in the target to the bodily fluid flow in a target control. In some cases, the differences in body fluid flow of a target and bodily fluid flow of target control may be indicative of disease. For example, in some cases, bodily fluid flow may be obstructed or reduced by a disease state, such as abnormal clotting, or blood vessel leakage, wherein the bodily fluid is blood. In some cases, a target control may be a healthy target. In some cases, a target control may be healthy tissue. In some cases, a target control may be a target that is known to be or suspected to be free of disease. In some cases, a target and target control may be in the same person, tissue or subject. In some cases, a target and target control may be in a different person, tissue or subject. In some cases, disease detection or stratification of treatment options may be performed wherein the bodily fluid flow is reduced as compared to a target control. In some cases, a disease detection or stratification of treatment options may be performed wherein the bodily fluid flow is increased as compared to a target control.

### E. Methods for Drug Screening and Development

The methods and systems of the disclosure provided can also investigate the efficacy of drugs on sample or test subject. Generally, methods and systems of fOCT objective focal length free flow measurement data may be used for platform screening of drugs, which may include either biologics or small molecules. In some examples, fOCT objective focal length free flow measurement data may be useful in determining the efficacy of a potential drug target which may be designed to increase or decrease a particular molecular maker or analyte in a target. For example, if a VEGF inhibitor is screened for use in the retina, fOCT objective focal length free flow measurement data may be used to assess candidate molecules for potential efficacy, toxicity and dosing by assessing the effect of the candidate molecules on blood flow in a particular area.

In some examples, a sample may include an in vitro cultured tissue graft, a harvested graft (e.g. from a cadaver, or an artificially grown tissue. In some examples, a test subject may include an animal, or genetically modified organism. In some examples, the genetically modified organism may exhibit one or more disease states or symptoms for which drug efficacy is tested. The provided method can also include high-throughput screening of FDA approved off-label drugs or experimental drugs.

### VI. Software and Computer Systems for fOCT

In various examples, the methods and systems of the present disclosure may further include software programs on computer systems and use thereof. Accordingly, computerized control for the synchronization of system functions such as laser system operation, fluid control function, and/or data acquisition steps are within the bounds of the invention. The computer systems may be programmed to control the timing and coordination of delivery of sample to a detection system, and to control mechanisms for diverting selected samples into a different flow path. In some examples, the computer may also be programmed to store the data received from a detection system and/or process the data for subsequent analysis and display.

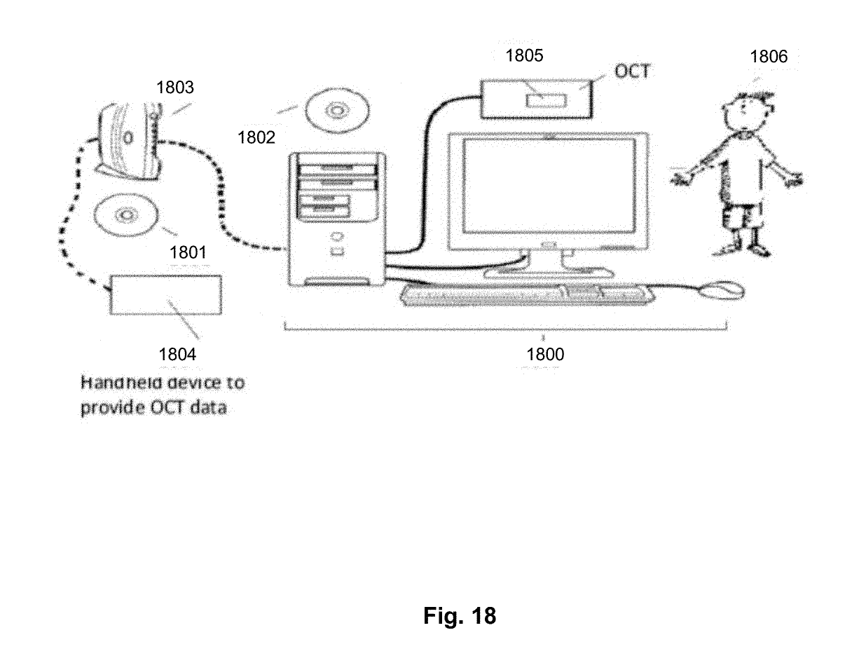

The computer system 1800 illustrated in FIG. 18 may be understood as a logical apparatus that can read instructions from media 1802 and/or a network port, which can optionally be connected to server 2503 having fixed media 1802. The system, such as shown in FIG. 18 can include a CPU, disk drives, optional input devices such as handheld devices for acquiring fOCT objective focal length free flow measurement data 1804 or other instrument types such as a laboratory or hospital based instrument 1805. Data communication can be achieved through the indicated communication medium to a server at a local or a remote location. The communication medium can include any suitable device for transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1806 as illustrated in FIG. 18.

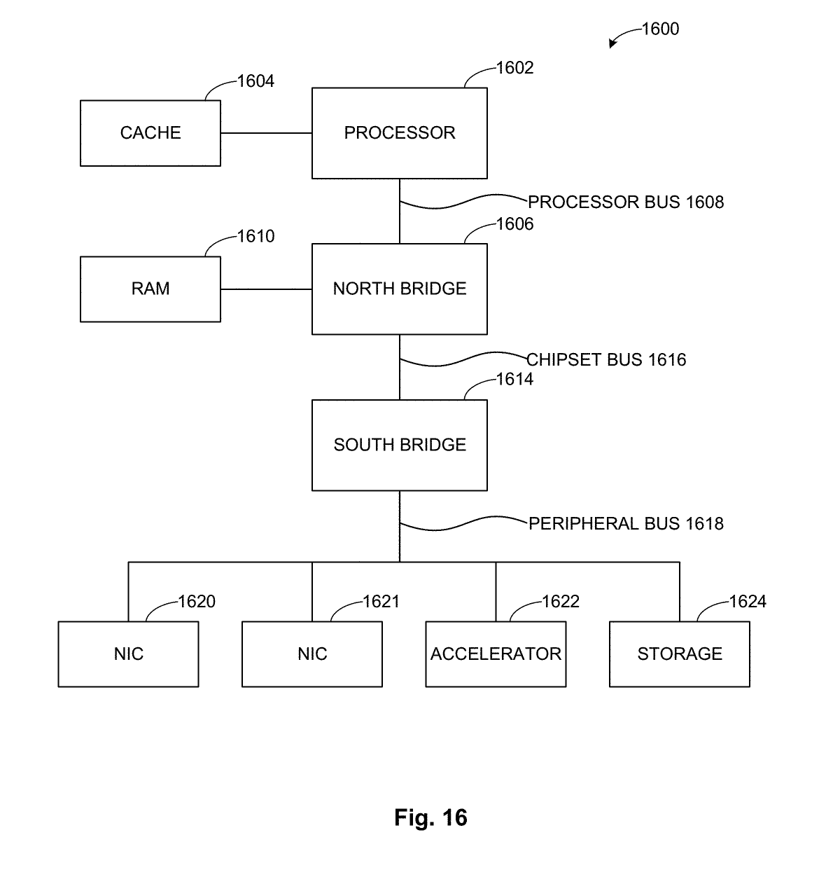

FIG. 1600 is a block diagram illustrating a first example architecture of a computer system 1600 that can be used in connection with the present disclosure. As depicted in FIG. 16, the example computer system can include a processor 1602 for processing instructions. Non-limiting examples of processors include: Intel Xeon™ processor, AMD Opteron™ processor, Samsung 32-bit RISC ARM 1176JZ(F)-S vl O™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some examples, multiple processors or processors with multiple cores can also be used, whether in a single computer system, in a cluster, or distributed across systems over a network comprising a plurality of computers, cell phones, and/or personal data assistant devices.

As illustrated in FIG. 16, a high speed cache 1604 can be connected to, or incorporated in, the processor 1602 to provide a high speed memory for instructions or data that have been recently, or are frequently, used by processor 1602. The processor 1602 is connected to a north bridge 1606 by a processor bus 1608. The north bridge 1606 is connected to random access memory (RAM) 1610 by a memory bus 1612 and manages access to the RAM 1610 by the processor 1602. The north bridge 1606 is also connected to a south bridge 1614 by a chipset bus 116. The south bridge 114 is, in turn, connected to a peripheral bus 1618. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 118. In some alternative architectures, the functionality of the north bridge can be incorporated into the processor instead of using a separate north bridge chip.

In some examples, system 1600 can include an accelerator card 1622 attached to the peripheral bus 118. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1624 and can be loaded into RAM 1610 and/or cache 104 for use by the processor. The system 1600 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with the present disclosure.

In this example, system 1600 also includes network interface cards (NICs) 1620 and 1621 connected to the peripheral bus for providing network interfaces to external storage, such as Network Attached Storage (NAS) and other computer systems that can be used for distributed parallel processing.

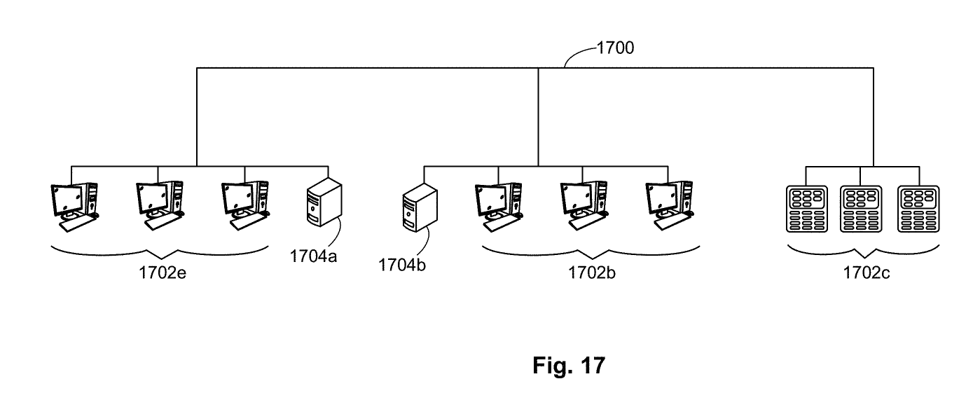

FIG. 17 is a diagram showing a network 1700 with a plurality of computer systems 1702a, and 1702b, a plurality of cell phones and personal data assistants 1702c, and Network Attached Storage (NAS) 1704a, and 1704b. In some examples, systems 1702a, 1702b, and 1702e can manage data storage and optimize data access for data stored in Network Attached Storage (NAS) 1704a and 1704b. A mathematical model can be used for the data and be evaluated using distributed parallel processing across computer systems 1702a, and 1702b, and cell phone and personal data assistant systems 1702c. Computer systems 1702a, and 1702b, and cell phone and personal data assistant systems 1702c can also provide parallel processing for adaptive data restructuring of the data stored in Network Attached Storage (NAS) 1704a and 1704b. FIG. 17 illustrates an example only, and a wide variety of other computer architectures and systems can be used in conjunction with the various examples of the present invention. For example, a blade server can be used to provide parallel processing. Processor blades can be connected through a back plane to provide parallel processing. Storage can also be connected to the back plane or as Network Attached Storage (NAS) through a separate network interface.

In some examples, processors can maintain separate memory spaces and transmit data through network interfaces, back plane or other connectors for parallel processing by other processors. In other examples, some or all of the processors can use a shared virtual address memory space.

The above computer architectures and systems are examples only, and a wide variety of other computer, cell phone, and personal data assistant architectures and systems can be used in connection with example examples, including systems using any combination of general processors, co-processors, FPGAs and other programmable logic devices, system on chips (SOCs), application specific integrated circuits (ASICs), and other processing and logic elements. In some examples, all or part of the computer system can be implemented in software or hardware. Any variety of data storage media can be used in connection with example examples, including random access memory, hard drives, flash memory, tape drives, disk arrays, Network Attached Storage (NAS) and other local or distributed data storage devices and systems.

In some examples, the computer system can be implemented using software modules executing on any of the above or other computer architectures and systems. In other examples, the functions of the system can be implemented partially or completely in firmware, programmable logic devices such as field programmable gate arrays, system on chips (SOCs), application specific integrated circuits (ASICs), or other processing and logic elements. For example, the Set Processor and Optimizer can be implemented with hardware acceleration through the use of a hardware accelerator card, such as accelerator card.

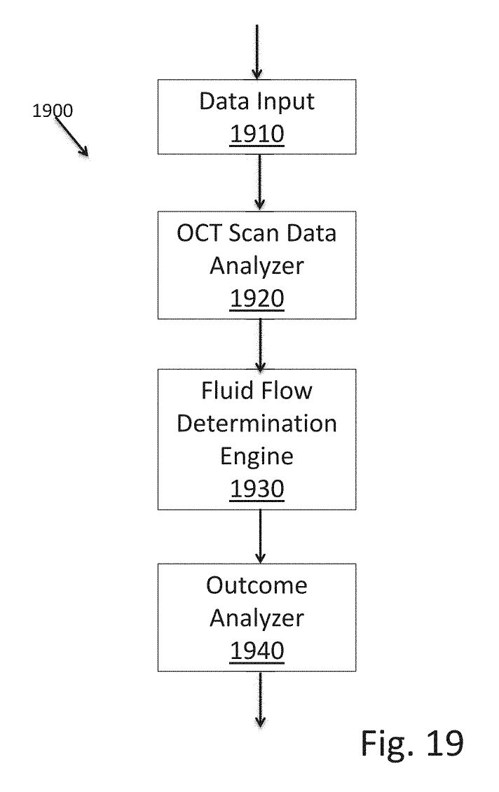

For example, as shown in FIG. 19, a fOCT data processing system 1900 includes a data input 1910 to receive OCT scan data from scanning of a target by an OCT device. One or more OCT scans may be generated and obtained by one or more components of the OCT device/system, such as a spectrometer (see, e.g., block 281 of example FIG. 2d). The example system 1900 includes an OCT scan data analyzer 1920 to process/analyze the OCT scan data according to one or more criterion, as described above (also see, e.g., block 284). The example system 1900 includes a fluid flow determination engine 1930 to determine fluid flow in the target based on the analysis from the data analyzer 1920 (see, e.g., block 285). Fluid flow information can be provided by the engine 1930 to an outcome analyzer 1940 to provide feedback and/or other output (e.g., display of information, printout of information, relay of information to another system (e.g., to drive another process), etc.). For example, the outcome analyzer 1940 can provide information to a healthcare practitioner and/or diagnostic system to facilitate a medical decision, such as described above (and also see, e.g., block 286).

### VII. Terminology

The terminology used therein is for the purpose of describing particular examples only and is not intended to be limiting of a device of this disclosure. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Several aspects of a device of this disclosure are described above with reference to example applications for illustration. It should be understood that numerous specific details, relationships, and methods are set forth to provide a full understanding of a device. One having ordinary skill in the relevant art, however, will readily recognize that a device can be practiced without one or more of the specific details or with other methods. This disclosure is not limited by the illustrated ordering of acts or events, as some acts may occur in different orders and/or concurrently with other acts or events. Furthermore, not all illustrated acts or events are required to implement a methodology in accordance with this disclosure.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another example. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5.

### EXAMPLES

### Example 1

This example describes a method to measure and analyze fOCT objective focal length free flow measurement of blood in a phantom experiment using an in vitro capillary tube. FIG. 6 shows the schematic diagram of the phantom experiment performed. One additional lens (either L6 or L7) was placed on the focal plane of the telescopic system (L4 and L5) to mimic the ocular lens. To simulate different objective focal lengths (e.g. eyeball axial lengths), the focal lengths of L6 and L7 were selected to be 10 mm and 12 mm, respectively, which focused the probing light onto a capillary tube (Paradigmoptics, CTPS125-250; inner diameter: 125 μm). The capillary tube was connected to a syringe pump (A-99, Razel). The Doppler angle was set on the capillary tube at approximately 82 degrees, which is close to the Doppler angle obtained from in vivo rat experiments. A 1% Intralipid suspension was pumped through the capillary tube, with flow rate changing from 1 μl/min to 8 μl/min. Eight consecutive pairs of small and large rings on the capillary tube were scanned, with the scanning angles β₁ and β₂ at four and six degrees, respectively. Each scanning ring contained 4,096 A-lines, and the A-line rate was 25-kHz. The maximum flow-induced mean phase shift in the phantom experiments was around 1.28 rad, which prevented phase wrap.

The example system used in this Example 1 experiment and Example 2 is described herein. A free-space SD-OCT was implemented, as shown in FIG. 6, where a supercontinuum laser (SuperK NKT photonics; center wavelength: 568.5 nm; bandwidth: 107 nm; spectrum of the laser source shown in FIG. 7a, and light was collimated and split into the sample arm and the reference arm. In the sample arm, the laser beam was scanned by a two-dimensional galvanometer (QS-7, Nutfield Technology) and relayed to the eye/phantom through a telescope system (f_{L4}=75 mm, f_{L5}=15 mm). The reflected light from the sample arm and the reference arm were combined and detected by a homemade spectrometer (spectral resolution: 0.0522 nm). The measured axial resolution of the SD-OCT was 1.7 μm in air, which provided 1.25 μm axial resolution retinal tissue, considering the refractive index of the retina is 1.36. The lateral resolution of the SD-OCT was about 15 μm. The imaging speed was 70 kHz for in vivo rat experiments and 25 kHz for phantom experiments. The signal-to-noise ratio (SNR) of the SD-OCT was measured at 95 dB with 70 kHz A-line rate. In the present study, the laser power was set at 0.8 mW for in vivo rat experiments, which is considered safe based on the American National Standards Institute for the safe use of lasers (ANSI Z136.1-2014).

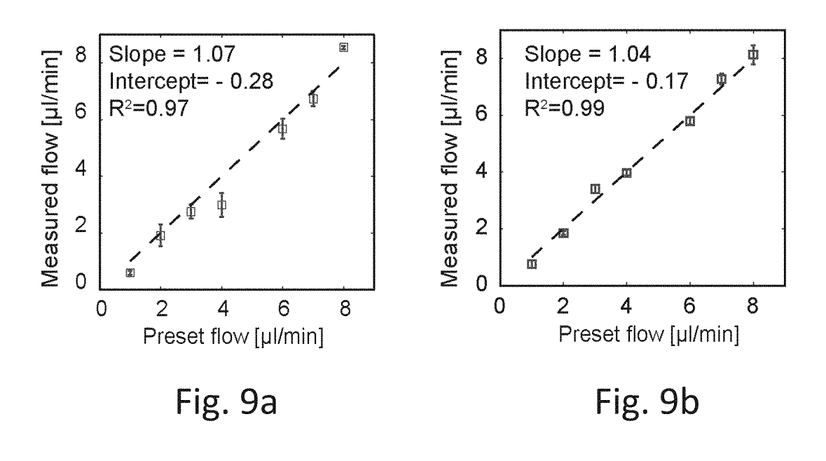

Two lenses with different focal lengths were used to mimic different eyeball axial lengths in the Intralipid flow phantom experiments. L6 had a focal length of 10 mm; L7 had a focal length of 12 mm. FIG. 9a shows that the measured flow rate was consistent with the preset values (linear fitting slope: 1.07; R²=0.97, where R² is the coefficient of determination) using L6. L6 with L7 were then replaced to test the stability of flow measurement without the eyeball size. The measured flow results are shown in FIG. 9b, in which the preset flow rates were consistent with measured flow rates (linear fitting slope: 1.04; R²=0.99). The accuracy in the Doppler angle measurement was further tested. Using L6, the detected Doppler angles were 82.4±0.4 and 84.6±0.45 degrees; using L7, the angles were 82.6±0.45 and 85±0.3 degrees. The measured Doppler angles were consistent with but slightly larger than the preset values (82 and 84 degrees).

### Example 2

In order to test fOCT objective focal length free flow measurements in a living target, Long Evans rats (Charles River, 300 g) were imaged in in vivo experiments. The rat was first anesthetized with a mixture of 2% isoflurane and 3 L/min regular air flow for ten minutes, then anesthetized with 1.5% isoflurane and 2 L/min regular air for five minutes. During imaging, the isoflurane rate and regular air flow rate were kept constant at 1.5% and 2 L/min, respectively. The rats' pupils were dilated with 1% tropicamide ophthalmic solution and paralyzed the iris sphincter muscles with 0.5% tetracaine hydrochloride ophthalmic solution. Artificial tear drops were applied (Systane, Alcon Laboratories) every minute to prevent corneal dehydration during imaging. The rats' electrocardiogram (ETH-256 Amplifier, Iworx) was also monitored during imaging. To measure the retinal blood flow, eight consecutive pairs of small and large rings (concentric rings) of beams of radiation were exposed on the rat retina, with scanning angles β₁ and β₂ at four and six degrees, respectively. To sample the pulsatile blood flow profile, all scanning rings (small and large) contained 4,096 A-lines at 70-kHz A-line rate.

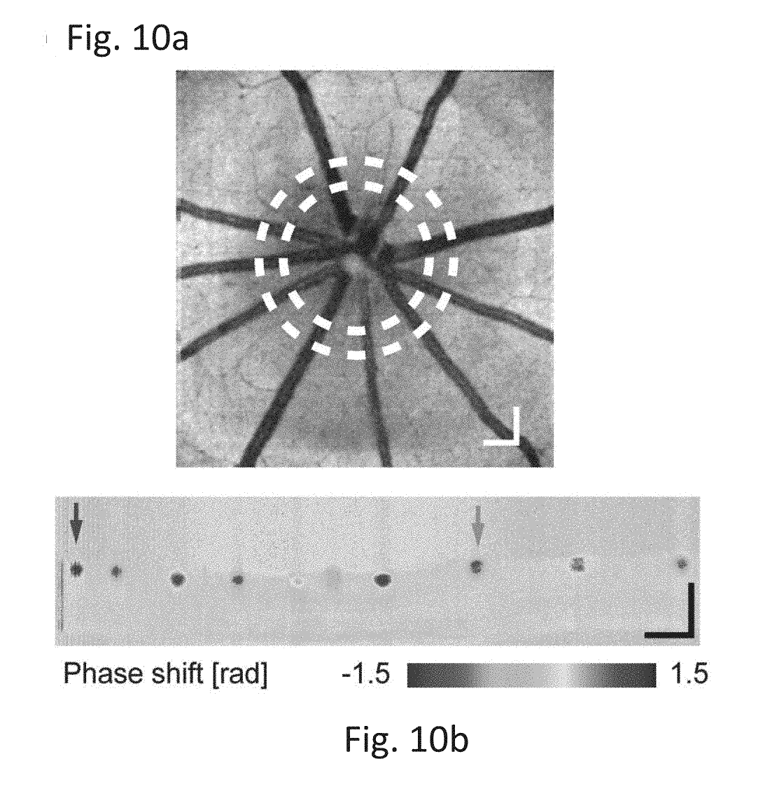

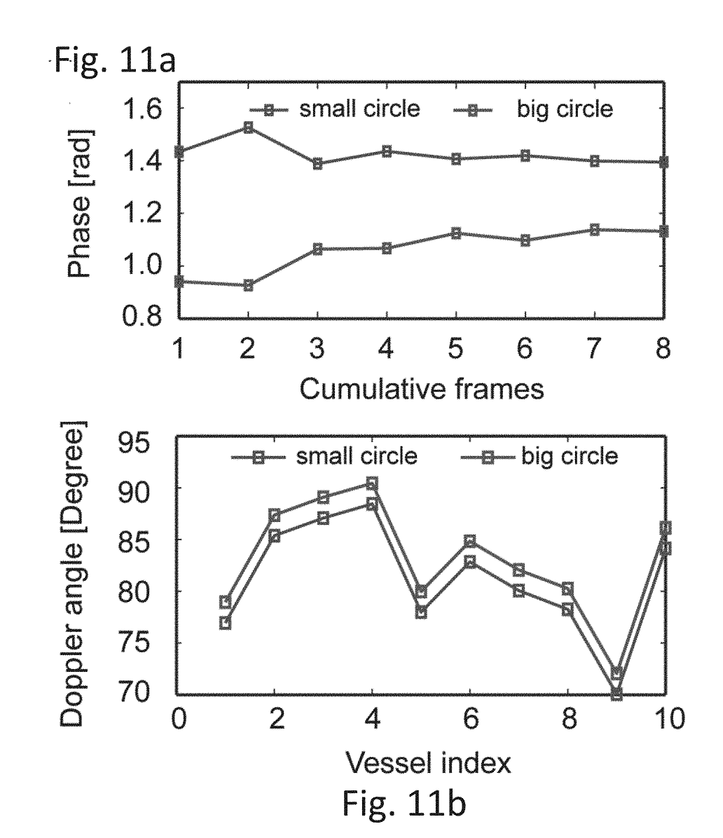

Flow quantification without eyeball axial length in rats in vivo was further tested. FIG. 10a shows the fOCT fundus image of one sample rat eye, where two white dashed circles highlight the trajectories of the dual-ring scanning. FIG. 10b shows one sample Doppler SD-OCT B-scan from the inner scanning ring; the red and blue arrows highlight an artery and a vein, respectively. Eight consecutive dual-ring pairs were used to scan the retina and the mean phase stability across a different number of cumulative Doppler B-scans was tested. The mean phase shifts ratio of the concentric rings was considered stable or substantially similar between the adjacent cumulative B-scans when there was difference less than 0.05 rad. In this example, the stable mean phase shift was achieved after seven B-scan data averaging (0.006 rad difference from sixth averaging to seventh averaging), as shown in FIG. 11a. With the stable phase shift from both small and large scanning rings, the Doppler angle θ was then retrieved (shown in FIG. 11b.

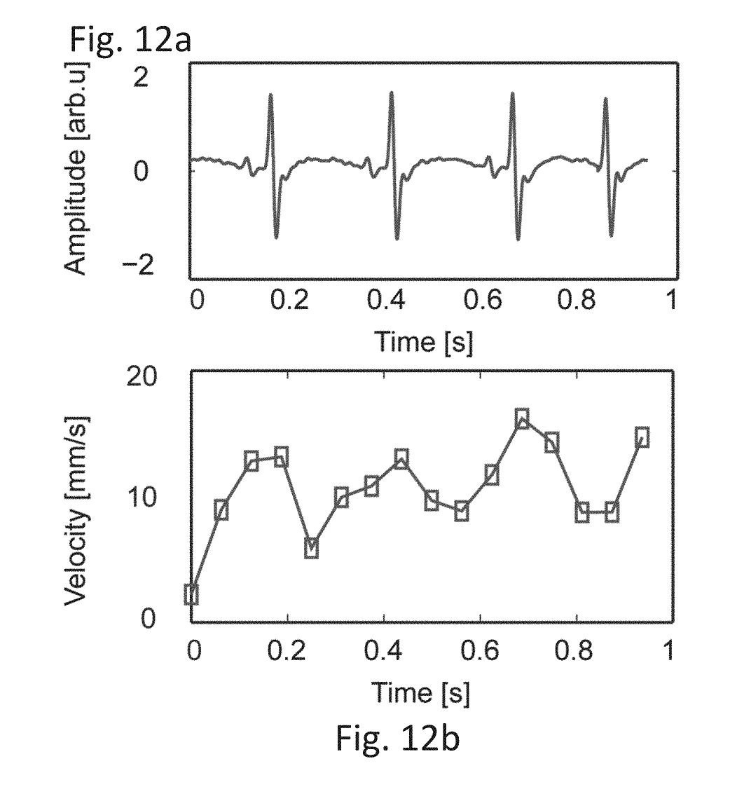

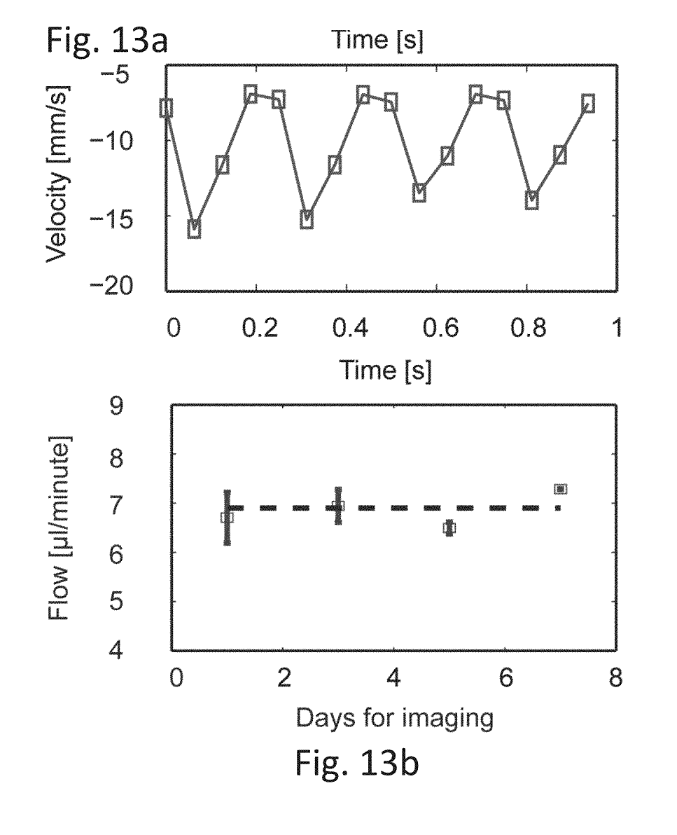

After extracting the Doppler angles, the pulsatile flow velocities were quantified the in both artery and vein. FIG. 12b and FIG. 13a show sample arterial and venous pulsatile flow velocities, respectively, where FIG. 12a shows the pulsatile amplitude. The retinal arterial and venous pulsatile blood flow were synchronized with the electrocardiogram, with the venous pulsatile flow profile delayed by 0.15 s. The absolute retinal flow rate was also quantified. In this particular rat, the total retinal blood flow was around 7 μl/min, which was consistent with other reported flows in rats under the same anaesthesia condition. The measured average total retinal blood flow was 7.02±0.31 μl/min among four different wild-type rats. Furthermore, the stability of the flow measurement without the eyeball axial length was confirmed when it was repeatedly monitored in the rats for one week. FIG. 13b shows that the four independent measuring retinal flow rate results were consistent: the standard deviation of the measured mean flow rate across four independent measuring points was 0.34 μl/min or 4.86%.

### Example 3

The ability to quantify retinal oxygen metabolic rate (rMRO₂) with fOCT can provide valuable insight into the pathogenesis of various retinal diseases, particularly DR and glaucoma. A key element is understanding the causal relationship between retinal cell degeneration and hemodynamic dysregulation. For example in DR, it is known that endothelial and pericyte disruption occurs in early-stage DR, but the hemodynamic changes that occur are unclear. Some studies showed increased retinal blood flow and suggested that the higher blood flow and high glucose level causes hyperperfusion, which further damages the endothelivam and pericytes; however, contradicting data exist that show decreased blood flow is one of the earliest changes in the diabetic retina. The hypothesis is that the loss of pericytes in the early phase of the disease reduces oxygen consumption, which may paradoxically lead to increased oxygenation of the retina. This might create a relative hyperoxia, resulting in vasoconstriction and reduced blood flow. Similarly, in glaucoma, there is degeneration of retinal ganglion cells and their axons. Although altered blood flow and vasculature were observed in glaucoma, their causal relationship to ganglion cell death remains unknown. A fOCT device or system configured for retinal scanning is setup to diagnose, monitor and treat patients for a variety of ophthalmic diseases. By generating fOCT eye axial length free flow measurement data, metabolic function is measured and related to a number of diseases where the retina experience a change in oxygen consumption as a result of disease or susceptibility to disease. The connection between hemodynamic dysregulation and retinal cell degeneration. With improved understanding of retinal metabolic function, improved approaches to early disease detection and therapeutic strategies can be designed.

### Example 4

In one example, a colonoscopy probe or endoscope is adapted for fOCT to evaluate the intestinal wall polyps for cancer. Currently, various other imaging techniques are used in conjunction with endoscopic imaging; however, the approach provides poor sensitivity and specificity. Yet, all cancers are known in the art to be highly vascular due to angiogenesis. Angiogenesis is a process of new blood vessel growth from preexisting blood vessels. Angiogenesis is a fundamental step of tumors from a dormant state to a malignant state, with new blood vessels penetrating into cancerous growths and supplying nutrients and oxygen. Since blood vessels carry hemoglobin, a fOCT enabled probe is able to provide highly accurate measurements of oxygen consumption as a function of fOCT objective focal length free flow measurements. Metabolic rate of one or more polyps is calculated as provided by the methods herein. Additionally, the fOCT probe is able to image with high resolution, various aspects of the vasculature underneath or around a polyp to help determine if the polyp may be pre-cancerous or cancerous at an earlier stage. It is generally known in the art that cancers have enhanced metabolic properties compared to normal tissues, so then cancerous cells have higher oxygen content from hemoglobin and a greater concentration of deoxygenated hemoglobin compared to normal tissues. Alternatively, when imaging potential colon cancer polyp with fOCT, comparing the fOCT images and fOCT objective focal length free flow measurements to what fOCT images and objective focal length free flow measurements of normal tissue; diagnosis is possible if increase blood vessel formation appears in the fOCT image or there is an increase is blood flow. Abnormal blood vessel formation could also be indicative of diseased tissue. For example, abnormal vascular patterns could be indicative of angiogenesis and putative colon cancer. Abnormal vascular patterns would be any vascular patterns outside the normal vasculature anatomy of the health colon tissue.

### Example 5

Another example of cancer diagnosis would include breast cancer. An fOCT probe is configured for in a needle and for a surgical tool for use in the removal of the breast cancer tumor. With the needle fOCT probe, the needle is to be placed at sites around the suspected area of the tumor to examine the morphology of the tissue and the tumor's vasculature. 3D fOCT images combined with fOCT objective focal length free flow measurement data of one or more areas of the breast help the surgeon determine optimal surgical margins for excision of the breast cancer tumor. Oxygenated hemoglobin molecules which have increased due to angiogenesis may be indicated to the surgeon by higher metabolic rate or an increase in flow measurement data as determined and calculated by fOCT methods. The cancerous cells in the breast with the higher oxygen content from hemoglobin and a greater concentration of deoxygenated hemoglobin could be imagined and diagnosed accordingly, when compared to normal breast tissue. Alternatively, when imaging the breast with fOCT, comparing the ultrasound image to what a fOCT image of normal tissue, diagnosis is possible if increased or abnormal blood vessel formation appears.

### Example 6

Another example of fOCT, includes configuring methods and devices for diagnostic techniques for diseased tissue with increased blood vessel formation, which could be detectable by with fOCT. Angiogenesis is known to occur during coronary artery disease, peripheral artery disease, and stroke when there's insufficient blood supply. For example, the blood vessels that surround large arteries or perfuse large arterial walls, such as vaso vasorum. These vessels surround the artery around the heart. If there is a plaque in these blood vessels, then the blood supply grows as the plaque size increases, and more cells from these additional blood vessels move into the plaque, making it unstable and more likely to rupture causing heart attacks and strokes. It has been shown that the endothelium of the vaso vasorum is disturbed in hypercholesterolemic conditions. This induces constriction of the vaso vasorun with subsequent lack of oxygen supply. Subsequently VEGF expression will increase with rapid vaso vasorum vessel formation as a consequence. In this example fOCT objective focal length free flow measurement data could obtained from inside blood vessels to measure blood flow in or around clots or thrombotic emboli which may lead to heart disease. Flow measurements may be taken to assess the seriousness of a plaque or the optimal place to perform an interventional procedure such as angioplasty or stenting.

### Example 7

In this example, fOCT is configured for a probe to be inserted into a catheter, which is directed to the site of an aneurysm. The fOCT probe is able to take successive measurements of the metabolic rate and objective focal length free flow measurement data of vessels in and around the aneurysm, informing the surgeon where to operate in an optimally safe place. The fOCT probe is used to guide one or more surgical instruments to the aneurysm site in need of treatment.

### Example 8

In this example, fOCT is configured for an intraoperative tool for use to analyze blood vasculature in the brain to help surgeons identify foci of abnormal neural activity. In the treatment of epilepsy, neuromodulation of one or more epileptic foci may be necessary to control epileptic symptoms. In order to identify foci, surgeons use the fOCT probe to identify regions in the brain with abnormal vasculature and increased metabolism from increased objective focal length free flow measurement data, which may correlate with abnormal neural activity associated with epilepsy. Using fOCT data, surgeons identify epileptic foci and apply treatment.

### Example 9

In this example, fOCT is used to monitor the treatment and prognosis of a patient with AMD. A patient presents symptoms of early stage AMD including the presence of drusen and sporadic blurriness and black patches in vision. A doctor administers Lucentis®, an FDA approved drug and anti-VEGF drug. The patient's retina is monitored and fOCT objective focal length free flow measurement data is obtained before and after administration of the drug. After 3 weeks, little to no effect is observed with Lucentis®. The doctor switches treatment and administers another anti-VEGF drug, Eyelea® to the patient. The patient's retina is monitored before and after administration of the drug Eyelea®.

What is claimed:

1. A method for imaging and quantifying fluid flow in a subject, the method comprising:
a. acquiring a first optical coherence tomography (OCT) data set for a first series of transverse locations in the subject, wherein the first data set comprises a first plurality of measurements, wherein at least two of the first plurality of measurements are made within a region substantially near a transverse location in the first series of transverse locations, wherein the first data set is acquired with a first beam of radiation having a first angle with respect to the subject;
b. acquiring second data set for a second series of transverse locations in the sample, wherein the second data set comprises a second plurality of measurements, wherein at least two measurements of the second plurality of measurements are made within a predetermined distance from the same transverse location as the at least two measurements of the first plurality of measurements, and wherein the second data is acquired with a second beam of radiation having a predetermined second angle different than the first angle;
c. determining axial fluid flow components from the first and second pluralities of measurements in both the first second data sets;
d. calculating the fluid flow within the sample based on a combination of the determined axial fluid flow components and without using predetermined objective focal lengths for the first and second beams of radiation; and
e. outputting results of the calculation for at least one of storage or display.

2. The method of claim 1 further including:
a. determining a vessel cross sectional area for the first and second beams of radiation;
b. determining an axial mean velocity for each data set over the vessel cross sectional area for the first and second beams of radiation.
c. calculating a mean velocity ratio over the vessel cross sectional areas for the first and second data sets for the first and second beams of radiation using the determined axial velocity components; and
d. calculating the flow using the ratio of the first and second mean velocities for the first and second data sets, multiplying by the cross sectional areas, and dividing by an angle difference between the first and second beams of radiation.

3. The method of claim 3, wherein the OCT system is a phase Doppler OCT system and the axial flow components are determined by calculating phase differences between the two or more measurements taken within a region substantially near a transverse location in first and second data sets.

4. The method of claim 1, wherein the objective focal lengths of the first and second beams of radiation is the axial length of an eyeball.

5. The method of claim 1, wherein the first and second data sets are acquired sequentially.

6. The method of claim 1, wherein the first and second data sets are acquired simultaneously.

7. The method of claim 1, wherein the first and second data sets are acquired using one or more beams of radiation configured in a predetermined shape.

8. The method of claim 1, wherein the first and second data sets are acquired using one or more beams of radiation configured as concentric circular patterns.

9. The method of claim 2, wherein the angle difference between the first angle and the second angle is chosen such that the signal-to-noise ratio of the phase shifts between the first and second beams of radiation are substantially similar.

10. The method of claim 2, wherein the angle difference between the first and second beams of radiation is chosen such that the depth position of the first and second beams of radiation are substantially similar.

11. A method for the diagnosis or treatment of a disease in a subject, the method comprising:
a. obtaining functional optical coherence tomography (fOCT) scans of a target using first and second beams of radiation;
b. determining the flow of bodily fluid in the target from the fOCT scans generated at (a), wherein the determining does not involve an objective focal length but instead uses measurements obtained from the fOCT scans;
c. facilitating a medical decision based on the determining of the flow of the bodily fluid.

12. The method of claim 11, wherein the medical decision is based on comparing the flow of the bodily fluid in the target to a flow of bodily fluid in a target control.

13. The method of claim 11, wherein the facilitating a medical decision includes a stratification of treatment options.

14. An optical coherence tomography system configured to generate fOCT objective length free fluid flow measurements, the system comprising:
a. a light source emitting light that is split to illuminate a target and illuminate a reference mirror;
b. a mirror to reflect the emitted light;
c. a detector to receive the emitted and reflected light; and
d. a processor to process received light from the detector to:
i. acquire a first optical coherence tomography (OCT) data set for a first series of transverse locations in the subject, wherein the first data set includes a first plurality of measurements, wherein at least two of the first plurality of measurements are made within a region substantially near a transverse location in the first series of transverse locations, wherein the first data set is acquired with a first beam of radiation having a first angle with respect to the subject;
ii. acquire a second data set for a second series of transverse locations in the sample, wherein the second data set comprises a second plurality of measurements, wherein at least two measurements of the second plurality of measurements are made within a predetermined distance from the same transverse location as the at least two measurements of the first plurality of measurements, and wherein the second data is acquired with a second beam of radiation having a predetermined second angle different than the first angle;
iii. determine axial fluid flow components from the first and second pluralities of measurements in both the first second data sets;
iv. calculate the fluid flow within the sample based on a combination of the determined axial fluid flow components and without using predetermined objective focal lengths for the first and second beams of radiation; and
v. output results of the calculation.

15. The system of claim 14, wherein the processor is further configured to:
a. determine a vessel cross sectional area for the first and second beams of radiation;
b. determine an axial mean velocity for each data set over the vessel cross sectional area for the first and second beams of radiation.
c. calculate a mean velocity ratio over the vessel cross sectional areas for the first and second data sets for the first and second beams of radiation using the determined axial velocity components; and
d. calculate the flow using the ratio of the first and second mean velocities for the first and second data sets, multiplying by the cross sectional areas, and dividing by an angle difference between the first and second beams of radiation.

16. The system of claim 15, wherein the OCT system is a phase Doppler OCT system and the axial flow components are determined by calculating phase differences between the two or more measurements taken within a region substantially near a transverse location in first and second data sets.

17. The system of claim 14, wherein the objective focal lengths of the first and second beams of radiation is the axial length of an eyeball.

18. The system of claim 14, wherein the first and second data sets are acquired sequentially.

19. The system of claim 14, wherein the first and second data sets are acquired simultaneously.

20. The system of claim 14, wherein the first and second data sets are acquired using one or more beams of radiation configured in a predetermined shape.

21. The system of claim 14, wherein the first and second data sets are acquired using one or more beams of radiation configured as concentric circular patterns.

22. The system of claim 15, wherein the angle difference between the first angle and the second angle is chosen such that the signal-to-noise ratio of the phase shifts between the first and second beams of radiation are substantially similar.

23. The system of claim 15, wherein the angle difference between the first and second beams of radiation is chosen such that the depth position of the first and second beams of radiation are substantially similar.
